(19) United States
(12) Patent Application Publication
(10) Pub. No.: US 2024/0279264 A1
(43) Pub. Date: Aug. 22, 2024

(54) CATALYTIC POLYMERIZATION AND DEPOLYMERIZATION OF 1,6-ANHYDROSUGARS

(71) Applicant: The Trustees of Boston College, Chestnut Hill, MA (US)

(72) Inventors: Jia NIU, Lexington, MA (US); Lianqian WU, Brington, MA (US)

(73) Assignee: The Trustees of Boston College, Chestnut Hill, MA (US)

(21) Appl. No.: 18/616,904

(22) Filed: Mar. 26, 2024

Related U.S. Application Data
(63) Continuation of application No. PCT/US2022/078036, filed on Oct. 13, 2022.
(60) Provisional application No. 63/270,638, filed on Oct. 22, 2021.

(51) Int. Cl. C07H 3/10; C07H 1/00; C08J 11/10

(52) U.S. Cl. CPC C07H 3/10; C07H 1/00; C08J 11/10; C08J 2305/00

(57) ABSTRACT

Methods for controlled synthesis and chemical recycling of stereoregular polysaccharides via polymerization of biomass-derived anhydrosugars are provided. The anhydrosugars react with an initiator in the presence of a synergy of a catalyst and the initiator providing the polysaccharides with high chain end fidelity and an excellent molecular weight distribution in a controlled manner, which allows for the preparation of polymeric systems with well-defined architectures. The polysaccharides obtained by such methods can be substantially converted to the start monomers in the present of a catalyst and can be used for biomedicine.

### CROSS-REFERENCE

This application is a continuation of a PCT International Application No. PCT/US2022/078036, filed on Oct. 13, 2022, which claims the benefit and priority of U.S. Provisional Application No. 63/270,638, filed Oct. 22, 2021, the entire content of each is incorporated herein by reference.

### STATEMENT REGARDING FEDERALLY SPONSORED RESEARCH OR DEVELOPMENT

This invention was made with government support under grant number CHE2117246 awarded by the National Science Foundation. The government has certain rights in the invention.

### BACKGROUND

Polysaccharides are among the most abundant biopolymers on Earth. In addition to their essential functions in biology¹, the utilization of polysaccharide-based biomass in renewable energy² and sustainable materials³ is also critical to reducing carbon emissions worldwide. However, polysaccharides isolated from biological sources are often heterogeneous⁴, imposing significant constraints on their characterization and utilization for emerging technologies. Therefore, the development of synthetic approaches to polysaccharides has been one of the main research areas in glycoscience⁵. For example, enzymatic synthesis has emerged as a promising method for polysaccharide synthesis owing to the perfect regio- and stereoselectivity of the enzymatic glycosylation, and the elimination of the need for protecting groups⁶. However, limited enzyme availability and high specificity generally restrict them to specific substrates or reactions. Iterative assembly strategies have been developed to prepare synthetic polysaccharides with precise sequence and length⁷. Notably, the automated glycan assembly (AGA) techniques developed by Seeberger has enabled the construction of complex polysaccharides (FIG. 1a)⁸⁻⁹. Nevertheless, the stepwise assembly methods require significant investments in equipment and reagents, and often have modest scalability.

Chemical polymerization is an efficient method for the scalable synthesis of polysaccharides and glycomimetic polymers¹⁰. Over the last ten years, many carbohydrate-derived monomers have been used to generate carbohydrate polymers, such as isosorbide¹¹, xylose¹², isohexide¹³, and levoglucosenyl ether¹⁴ (FIG. 1a). Particularly worth noting are the living anionic ring-opening polymerization of monosaccharide-derived cyclic carbonate and b-lactam reported by Wooley¹⁵ and Grinstaff¹⁶, respectively. However, these polymers all consist of non-glycosidic linkages, and in some cases, control over the polymerization remains challenging. While the cationic ring-opening polymerization (CROP) of 1,6-anhydrosugars has been employed to produce polysaccharides since the 1960s¹⁷, achieving control over this polymerization remains a formidable challenge.

Synthetic polymers play an essential role in the modern human society due to their low cost, high stability, and excellent material properties. However, they impose increasingly serious environmental issue, caused by unsustainable generation (˜320 Mt in 2015) and disposal of single-used commodity plastics, pushing people to find numerous approaches to address this challenge from the perspective of circular economy. Among them, chemical recycling via depolymerization to their constituent monomer, where polymer-to-polymer closed-loop recycling would be achieved, represents an ideal solution to the end-of-use issue of plastics. Although tremendous advances have been made in the last decades, the chemical recycling approach still faces challenges, such as, relatively complicated monomer syntheses, energy-intensive polymerization conditions (typically low temperature) and uncomplete recycling of the precursor motif chemicals. In addition, the material properties of the virgin and recycled polymers, including thermostability, crystallinity and mechanical behaviors, must be concerned as well.

Thus, there is a need to develop chemically recyclable polymers with novel structures and encouraging properties from more environmentally friendly biomass-derived feedstocks. Polysaccharides with inherent structural rigidity and stereoregularity prognosticate remarkable stability and crystallinity, as well as other unexpected material properties. In addition, not only can polysaccharides be readily prepared from abundant and sustainable natural monosaccharides, but also their potentially labile glycosylic bond will help to unzip the polymer chain to achieve complete chemical recycling. Hence, extensive efforts have been made to pursue efficient polysaccharides synthesis. State-of-the-art iterative glycosylation has been widely used to construct complex polysaccharides, and glycans more than 100-mer have been successfully obtained by Seeberger and Yu lab, but tedious protection/deprotection steps used therein offset the benefits associated with the renewable feedstocks. Polymerization represent another powerful strategy to prepare polysaccharides, despite free from lab-consuming tasks, but poor control over the polymerization hamper their application in the precision polysaccharides synthesis. The present disclosure provides efficient synthetic routes to precision polysaccharides to satisfy these needs and other needs.

### SUMMARY

In accordance with the purpose(s) of the present disclosure, as embodied and broadly described herein, the disclosure provides a chemical approach to produce stereoregular polysaccharides from abundant natural resources of 1,6-anhydrosugars with precise control over molecular weights. The resulting polysaccharides possess excellent chemical stabilities against strong acid, base, and under high temperature, but can be readily recycled and depolymerized into the starting monomers in the presence of the catalyst. Existing strategies do not offer the level of control over polymer structures and molecular weights achieved by the method of the present disclosure and cannot achieve a circular life cycle demonstrated in the present disclosure.

In one aspect, the present disclosure provides a method of controlled synthesizing and chemical recycling of a stereoregular polysaccharide comprising reacting a biomass-derived anhydrosugar having Formula I:

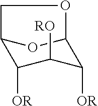

wherein R is a group selected from C1-C6 alkyl group, with an initiator in the presence of a synergy of a catalyst and the initiator. In certain embodiments, the R is a group selected from C1-C6 alkyl group, C1-C5 alkyl group, C1-C4 alkyl group, C1-C3 alkyl group, C1-C2 alkyl group, and/or a methyl group.

In certain embodiments, the initiator is glucosyl halide having a structure represented by the formula:

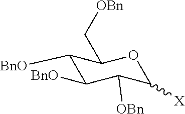

wherein the halide is a group selected from fluoride, chloride, bromide or iodide. The initiator provides the polysaccharide with high chain end fidelity and an excellent molecular weight distribution in a controlled manner. In certain embodiments, the polysaccharide of the present disclosure has 1-95% chain end fidelity, 50-95% chain end fidelity, or about 90%-95% chain end fidelity.

In certain embodiments, the catalyst is a Lewis acid, including but not limited to, Cu(OTf)₂, BF₃ or Boron trifluoride complex, such as boron trifluoride diethyl etherate.

The present disclosure further provides that the polysaccharides are made by the method described herein. In certain embodiments, the polysaccharide of the present disclosure has a molecular weight that ranges from 2.6 to 32.2 kDa. The polysaccharide of the present disclosure can replace polyethylene glycol (PEG), a widely used polymer in drug delivery applications ranging from anti-cancer drugs to mRNA vaccines.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Many aspects of the present disclosure can be better understood with reference to the drawings. The components in the drawings are not necessarily to scale, emphasis instead being placed upon clearly illustrating the principles of the present disclosure. Moreover, in the drawings, like reference numerals designate corresponding parts throughout the several views. A better understanding of the features and advantages of the invention will be obtained by reference to the following detailed description that sets forth illustrative embodiments, in which the principles of the invention are utilized, and the accompanying drawings (also “Figure” and “FIG.” herein), of which:

FIGS. 1a-1c. Living cationic ring-opening polymerization of 1,6-anhydrosugars. FIG. 1a. Established pathways to polysaccharides (Prior Art). FIG. 1b. Representative reaction scheme of living cationic ring-opening polymerization of 1,6-anhydrosugars in this work. FIG. 1c. Proposed mechanism. LG, leaving group; PG, protecting group; tPG, temporary protecting group; Bn, benzyl; Me, methyl; R, alkyl/allyl group.

FIGS. 2a-2f. Preparation and characterization of precision polysaccharides. FIG. 2a. Development of living CROP of 1,6-anhydrosugars. FIG. 2b. MALDI-TOF MS spectrum of P(Me-D-Glc). FIG. 2c. ¹H NMR spectra of P(Me-D-Glc). FIG. 2d. (i) Plots of Mₙ and Ð as a function of monomer conversion and (ii) Plots of Mₙ and Ð as a function of the [M]₀/[I]₀ ratio. FIG. 2e. Synthesis of block copolysaccharide P(ⁿBu-D-Glc)-b-P(Me-D-Glc). FIG. 2f, Chain end modification of P(Me-D-Glc). Et, ethyl; ⁿBu, n-butyl; Glc, glucose; OTf, trifluoromethanesulfonate.

FIGS. 3a-3b. Monomer scope and computational studies. FIG. 3a. Monomer scope: polymerization reactions were performed at 25° C. in dichloromethane for six hours; more experimental details are provided in Supplementary Information. FIG. 3b. Energy profile of reversible deactivation and propagation process. Free energies (kcal mol⁻¹) are obtained at the level of wB97X-2-D3(BJ)/ma-def2-TZVPP-def2-TZVPP/SMD(DCM)//B3LYP-D3(BJ)/ma-def2-SVP-def2-SVP/PCM(DCM). The DFT calculations were performed starting from a methyl-protected glucosyl fluoride 1 to mimic the dormant species during living CROP and simplify the calculations. ⁿPr, n-propyl; ⁿPen, n-pentyl; All, allyl.

FIGS. 4a-4c. Synthesis of biologically relevant precision polysaccharides. FIG. 4a. Synthesis of a-1,6-D-glucan P(OH-D-Glc) and comparison to natural a-1,6-D-glucan. FIG. 4b. Enzymatic degradation studies of P(OH^{Bn}-D-Glc) and P(OH^{Bn}-L-Glc). FIG. 4c. Synthesis of a-1,6-D-mannan P(OH-D-Man). Pd, Pd(PPh₃)₄ and Pd(OH)₂; Man, mannose.

FIGS. 5a-5e. Chemical recycling and material properties of precision polysaccharides. FIG. 5a. Polymerization-depolymerization cycles. FIG. 5b. (i) SEC trace overlays of P(Me-D-Glc) treated by various additives and (ii) TGA curves of P(R-D-Glc) (R=Me, Et, ⁿPr, ⁿBu, ⁿPen). FIG. 5c. (i) DSC curve and powder XRD profile (inset) of P(Me-D-Glc) and (ii) overlays of DSC curves of P(R-D-Glc) (R=Et, ⁿPr, ⁿBu, ⁿPen). FIG. 5d. Stress-strain curves of PN1 (green) and PN2 (magenta). FIG. 5e. Depolymerization of materials and reprepared materials PN1′ and PN2′ from recovered monomers.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE INVENTION

Petroleum-based plastics are currently produced in a massive scale worldwide and contribute to a significant portion of the plastic waste accumulation in the environment. While much effort has been made to develop sustainable and degradable polymers (e.g., polyesters and polylactides) as potential replacements for traditional vinyl plastics, they are still single-use plastics that cannot be chemically recycled. The present disclosure provides a promising solution to this challenge. Starting from 1,6-anhydrosugar, an abundant natural product, a novel class of polysaccharides can be generated with precisely controlled molecular weight and stereochemistry. This unprecedented capability is of immediate and broad interest to the polymer industry, which has seen a significant surge of interest in addressing the global crisis of plastic pollution.

Disclosed herein is a novel approach that can achieve polysaccharides with high chain end fidelity and an excellent molecular weight distribution via polymerization of biomass-derived anhydrosugars in a controlled manner. The resulting polysaccharides possess excellent chemical stabilities against strong acid, base, and under high temperature. As a significant conceptual advance, the novel approach described herein has, surprisingly, enabled a fully closed recycling loop for the biomass-derived anhydrosugars using the same catalysts both in the polymerization to obtain the polysaccharides and unzipping thereof.

In one aspect, the disclosed methods provides that the anhydrosugars react with an initiator in the presence of a synergy of a catalyst and the initiator achieving the polysaccharides with high chain end fidelity and an excellent molecular weight distribution in a controlled manner. In a further aspect, the polysaccharides obtained by such methods can be recycled and depolymerized into the starting monomers in the presence of the catalyst.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a monomer,” “a catalyst,” or “a polymer,” includes, but is not limited to, mixtures or combinations of two or more such monomers, catalysts, or polymers, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e., one atmosphere).

The composition, sequence, length, and type of glycosidic linkages of polysaccharides profoundly affect their biological and physical properties. However, investigation of the structure-function relationship of polysaccharides is hampered by accessing well-defined polysaccharides in sufficient quantities. Here, the present disclosure provides a chemical approach to precision polysaccharides with native glycosidic linkages via living cationic ring-opening polymerization of 1,6-anhydrosugars. In certain embodiments, well-defined polysaccharides with tunable molecular weight, low dispersity, and excellent regio- and stereoselectivity were synthesized using a boron trifluoride etherate catalyst and glycosyl fluoride initiators. Computational studies revealed that the reaction propagated through the monomer a-addition to the oxocarbenium and was controlled by the reversible deactivation of the propagating oxocarbenium to form the glycosyl fluoride dormant species. The method disclosed herein afforded a facile and scalable pathway to multiple biologically relevant precision polysaccharides, including D-glucan, D-mannan, and an unusual L-glucan. It is demonstrated that catalytic depolymerization of precision polysaccharides efficiently regenerated monomers, suggesting their utility as a class of chemically recyclable materials with tailored thermal and mechanical properties.

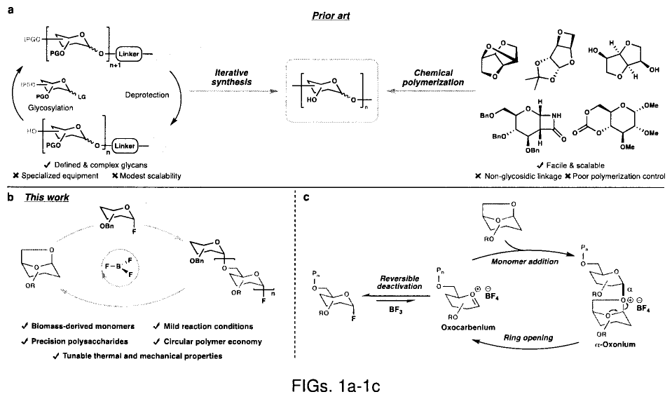

In certain embodiments, the synergistic combination of a glycosyl donor initiator and a mild Lewis acid catalyst were leveraged to achieve living CROP of 1,6-anhydrosugars (FIG. 1b). Native polysaccharides with controlled molecular weight and excellent regio- and stereoselectivity could be readily prepared from these biorenewable monomers. The mechanism of the disclosed method is centered on reversible deactivation of the propagating oxocarbenium¹⁸, which is generated by the fluoride abstraction from glycosyl fluoride initiator by boron trifluoride catalyst. Addition of the 1,6-anhydrosugar monomer to oxocarbenium from the less sterically demanding a-face affords an a-oxonium¹⁹. Followed by a rapid ring opening process, the oxocarbenium was regenerated. An equilibrium between oxocarbenium species and dormant glycosyl fluoride is established to achieve controlled chain growth (FIG. 1c). It is worth noting that Coates et al. recently harnessed a similar strategy to accomplish the controlled polymerization of cyclic acetals²⁰.

The present disclosure provides a chemical approach to precision native polysaccharides through living polymerization of 1,6-anhydrosugars. The generality of this method enabled the facile synthesis of a variety of polysaccharides and oligo-glycans with excellent regio- and stereoselectivity, precise molecular weight control, and high fidelity of the chain end groups. In addition, the obtained materials displayed excellent chemical recyclability and tunable thermal and mechanical properties. Overall, the method disclosed herein created a new paradigm for the chemical synthesis of polysaccharides with strong implications in a range of applications spanning from materials science to bioengineering.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Materials and Methods

All manipulations of air- and moisture-sensitive materials were carried out under nitrogen in a glovebox or by using standard Schlenk line techniques. Solvents for polymerization, such as Dichloromethane (DCM), chloroform (CHCl₃) and acetonitrile (CH₃CN), were dried over calcium hydride for 24 hrs, distillated to an oven-dried Schlenk flask, and stored in a nitrogen gas-filled glovebox. Solvents for air sensitive reactions, such as tetrahydrofuran (THF), dimethylformamide (DMF), were purchased from Fisher Scientific and used after purification by a dry solvent system (Pure Process Technology). Otherwise, solvents (hexane, acetone, toluene, EtOAc, MeOH, DMSO) were used as received. BF₃·Et₂O was dried over calcium hydride for 24 hrs, vacuum distillated to an oven-dried Schlenk flask, and stored under nitrogen in a glovebox freezer at −30° C. All other chemicals were purchased from Alfa Aesar, Sigma-Aldrich, Acros, Fisher Scientific, or TCI chemical companies and used as received without further purification.

¹H, ¹³C, ¹⁹F NMR—Spectra were recorded on either a Varian Gemini-600 (600 MHz) or Varian Inova-500 (500 MHZ) NMR spectrometer Chemical shifts (δ) for ¹H and ¹³C NMR spectra were referenced to protons of the residual solvent (for ¹H) or deuterated solvent itself (for ¹³C).

Powder X-Ray Diffraction (PXRD)—Analyses were performed on a Rigaku MiniFlex diffractometer. The voltage was set to 40 kV, and the current was 40 mA. The detector collected data over the range 2θ=5-50°.

Specific Rotation—Analyses were performed on a Rudolph Analytical Research Autopol IV Polarimeter. The wavelength of the light used is 589 nanometers (the sodium D line), and the path length is 0.5 dm. The data was collected at 20° C.

High-resolution mass spectrometry (HRMS)—Analyses were performed on JEOL AccuTOF DART Micromass LCT ESI-MS.

Matrix-Assisted Laser Desorption/Ionization Time-of-Flight Mass Spectrometry (MALDI-TOF MS)—Analyses were performed on a Bruker Auto Flex Max instrument (positive mode). α-Cyano-4-hydroxycinnamic acid was used as matrix with NaI added as the cation source.

Size-exclusion chromatography (SEC)—Analyses were carried out using a Tosoh's high performance SEC system HLC-8320GPC with a refractive index detector. The Tosoh SEC system was equipped with two TSKgel GMH_{HR}-N columns (5 μm, 7.8 mm ID), which were eluted with CHCl₃ at 40° C. at a rate of 0.5 mL/min and calibrated using polystyrene standards (ReadyCal Kit, Sigma-Aldrich #81434). Deprotected polymer molecular weights were determined by SEC using DMF (0.01 M LiBr) as the eluent at a flow rate of 0.5 mL/min through three TSKgel AlphaM columns (13 μm, 7.8 mm ID) at 50° C. with a refractive index detector.

Thermogravimetric Analysis (TGA)—Thermal gravimetric analysis was obtained using a Netzsch Instruments STA 449 F1 Jupiter. Analysis was performed on ˜10 mg of a given sample at a heating rate of 10° C./min from 25 to 600° C. under nitrogen gas.

Differential Scanning calorimetry (DSC)—Data was recorded on a Netzsch instruments DSC 214 Polyma using ˜10 mg samples. All Tₘ and T_{g} values were obtained from a second scan after the thermal history was removed from the first scan. The second heating rate was 10° C./min and cooling rate was 10° C./min.

Uniaxial Tensile Elongation Tests—Analyses were performed on an Instron 5567 universal testing system equipped with a 1 kN load cell and manual grips, at a crosshead speed of 5 mm/min. A pre-strain of 0.1 N was also applied. The samples were prepared in Teflon molds with cavity dimensions of (63.5×9.5×3.2 mm³). Grip separation was used as the gauge length for the purposes of calculating strain. Extension to failure was performed using three parallel samples and the data were analyzed using the Bluehill Universal Software. The Young's modulus was calculated using the slope of the stress-strain curve from 0 to 1% strain.

Monomer Synthesis

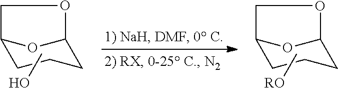

General procedure: To a suspension of NaH in DMF was added 1,6-anhydro-β-D-glucopyranose at 0° C., and the mixture was stirred for additional 30 mins. Subsequently, alkyl halide was added slowly at 0° C. After being allowed to warm up to 25° C., the reaction was left stirring for 12 hrs. The reaction was quenched with MeOH, and the solvent was removed under reduced pressure. DCM was added, successively washed with water and brine, and then dried over Na₂SO₄. After filtration, all the volatiles were removed under vacuum and the crude was purified by flash chromatography to afford the desired product.

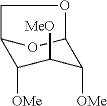 [SMILES: COC1C2COC(O2)C(OC)C1OC]

Prepared according to the general procedure using 1,6-anhydro-β-D-glucose (10 g, 61.7 mmol, 1.0 equiv), NaH (8.6 g, 215.9 mmol, 3.5 equiv), methyl iodide (13.4 mL, 215.9 mmol, 3.5 equiv) in DMF (150 mL) at 25° C. The crude was purified by flash chromatography (hexane/ethyl acetate=2/1) to afford the desired product Me-D-Glc as a white solid (11.8 g, 94% yield). ¹H NMR spectra is consistent with literature data⁴¹. ¹H NMR (500 MHZ, CDCl₃): δ 5.47 (s, 1H), 4.62 (dd, J=6.0, 1.6 Hz, 1H), 3.91 (dd, J=7.2, 0.8 Hz, 1H), 3.72 (dd, J=7.2, 6.0 Hz, 1H), 3.47 (s, 3H), 3.46 (s, 3H), 3.44 (s, 3H), 3.33-3.31 (m, 1H), 3.14-3.12 (m, 1H), 3.10-3.08 (m, ¹H).

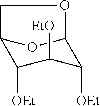 [SMILES: CCOC1C2COC(O2)C(OCC)C1OCC]

Prepared according to the general procedure using 1,6-anhydro-β-D-glucose (10 g, 61.7 mmol, 1.0 equiv), NaH (8.6 g, 215.9 mmol, 3.5 equiv), ethyl iodide (17.3 mL, 215.9 mmol, 3.5 equiv) in DMF (150 mL) at 25° C. The crude was purified by flash chromatography (hexane/ethyl acetate=6/1) to afford the desired product Et-D-Glc as a colorless liquid (14.0 g, 92% yield). ¹H NMR (600 MHZ, CDCl₃): δ 5.38 (s, 1H), 4.54 (dd, J=6.0, 1.4 Hz, 1H), 3.88 (dd, J=7.0, 1.4 Hz, 1H), 3.69-3.66 (m, 1H), 3.64-3.55 (m, 6H), 3.38-3.36 (m, 1H), 3.19-3.17 (m, 1H), 3.16-3.13 (m, 1H), 1.24-1.17 (m, 9H). ¹³C NMR (125 MHZ, CDCl₃): δ 100.57, 78.46, 77.87, 77.28, 74.22, 65.54, 65.35, 65.27, 64.69, 15.24, 15.17, 15.15. HRMS (DART-MS) m/z calcd. for C₁₂H₂₃O₅ [M+H]⁺ 247.15400. found: 247.15431.

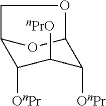

Prepared according to the general procedure using 1,6-anhydro-β-D-glucose (2.0 g, 12.3 mmol, 1.0 equiv), NaH (2.2 g, 55.5 mmol, 4.5 equiv), 1-iodopropane (5.4 mL, 55.5 mmol, 4.5 equiv) in DMF (70 mL) at 50° C. The crude was purified by flash chromatography (hexane/ethyl acetate=9/1) to afford the product ⁿPr-D-Glc as a colorless liquid (2.1 g, 62% yield). ¹H NMR (600 MHZ, CDCl₃): δ 5.40 (s, 1H), 4.56 (dd, J=6.0, 1.4 Hz, 1H), 3.89 (dd, J=7.0, 1.4 Hz, 1H), 3.69 (dd, J=7.0, 6.0 Hz, 1H), 3.55-3.45 (m, 6H), 3.38-3.36 (m, 1H), 3.19-3.17 (m, 1H), 3.16-3.13 (m, 1H), 1.66-1.56 (m, 6H), 0.95-0.91 (m, 9H). ¹³C NMR (125 MHZ, CDCl₃): δ 100.86, 79.04, 78.32, 77.49, 74.54, 72.19, 71.87, 71.35, 65.65, 23.14, 23.02, 23.00, 10.61, 10.56, 10.51. HRMS (DART-MS) m/z calcd. for C₁₅H₂₉O₅ [M+H]⁺ 289.20095. found: 289.20084.

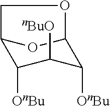

Prepared according to the general procedure using 1,6-anhydro-β-D-glucose (2.0 g, 12.3 mmol, 1.0 equiv), NaH (2.2 g, 55.5 mmol, 4.5 equiv), 1-iodobutane (6.3 mL, 55.5 mmol, 4.5 equiv) in DMF (70 mL) at 50° C. The crude was purified by flash chromatography (hexane/ethyl acetate=12/1) to afford the product ⁿBu-D-Glc as a colorless liquid (2.4 g, 59% yield). ¹H NMR (600 MHZ, CDCl₃): δ 5.40 (s, 1H), 4.54 (dd, J=5.8, 1.4 Hz, 1H), 3.88 (dd, J=7.0, 1.2 Hz, 1H), 3.69 (dd, J=7.0, 5.8 Hz, 1H), 3.60-3.49 (m, 6H), 3.37-3.34 (m, 1H), 3.18-3.16 (m, 1H), 3.15-3.13 (m, 1H), 1.61-1.53 (m, 6H), 1.42-1.36 (m, 6H), 0.94-0.90 (m, 9H). ¹³C NMR (150 MHZ, CDCl₃): δ 100.85, 79.03, 78.29, 77.46, 74.53, 70.26, 69.90, 69.40, 65.65, 32.01, 31.87, 31.85, 19.33, 19.27, 19.21, 13.84, 13.82. HRMS (DART-MS) m/z calcd. for C₁₈H₃₅O₅ [M+H]⁺ 331.24790. found: 331.24691.

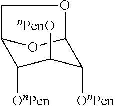

Prepared according to the general procedure using 1,6-anhydro-β-D-glucose (2.0 g, 12.3 mmol, 1.0 equiv), NaH (2.2 g, 55.5 mmol, 4.5 equiv), 1-bromopentane (6.9 mL, 55.5 mmol, 4.5 equiv) in DMF (70 mL) at 50° C. The crude was purified by flash chromatography (hexane/ethyl acetate=15/1) to afford the product ⁿPen-D-Glc as a colorless liquid (3.3 g, 71% yield). ¹H NMR (500 MHZ, CDCl₃): δ 5.40 (s, 1H), 4.54 (dd, J=5.8, 1.4 Hz, 1H), 3.87 (dd, J=7.0, 1.4 Hz, 1H), 3.69 (dd, J=7.0, 5.8 Hz, 1H), 3.59-3.48 (m, 6H), 3.37-3.34 (m, 1H), 3.18-3.16 (m, 1H), 3.15-3.13 (m, 1H), 1.63-1.56 (m, 6H), 1.36-1.31 (m, 12H), 0.92-0.88 (m, 9H). ¹³C NMR (150 MHz, CDCl₃): δ 100.86, 79.07, 78.36, 77.51, 74.55, 70.62, 70.25, 69.75, 65.67, 29.62, 29.51, 29.49, 28.34, 28.29, 28.22, 22.48, 22.46, 14.00, 13.98. HRMS (DART-MS) m/z calcd. for C₂₁H₄₁O₅ [M+H]⁺ 373.29485. found: 373.29423.

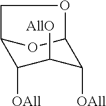

Prepared according to the general procedure using 1,6-anhydro-β-D-glucose (1.6 g, 10 mmol, 1.0 equiv), NaH (1.4 g, 35.0 mmol, 3.5 equiv), allyl bromide (3.0 mL, 35.0 mmol, 3.5 equiv) in DMF (20 mL) at 25° C. The crude was purified by flash chromatography (hexane/ethyl acetate=6/1) to afford the product All-D-Glc as a colorless liquid (2.7 g, 96% yield). ¹H NMR spectra is consistent with literature data⁴². ¹H NMR (500 MHZ, CDCl₃): δ 5.98-5.86 (m, 3H), 5.43 (s, 1H), 5.33-5.27 (m, 3H), 5.22-5.19 (m, 3H), 4.58 (dd, J=5.8, 1.4 Hz, 1H), 4.14-4.09 (m, 6H), 3.92 (dd, J=7.2, 1.2 Hz, 1H), 3.70 (dd, J=7.2, 5.8 Hz, 1H), 3.52-3.50 (m, 1H), 3.32-3.30 (m, 1H), 3.28-3.26 (m, ¹H).

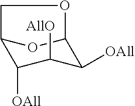

Prepared according to the general procedure using 1,6-anhydro-β-D-mannose (1.0 g, 6.2 mmol, 1.0 equiv), NaH (0.87 g, 21.6 mmol, 3.5 equiv), allyl bromide (1.9 mL, 21.6 mmol, 3.5 equiv) in DMF (15 mL) at 25° C. The crude was purified by flash chromatography (hexane/ethyl acetate=4/1) to afford the product All-D-Man as a colorless liquid (1.5 g, 86% yield). ¹H NMR (500 MHZ, CDCl₃): δ 5.97-5.87 (m, 3H), 5.44 (s, 1H), 5.32-5.17 (m, 6H), 4.52 (d, J=5.8, Hz, 1H), 4.21 (d, J=7.0, Hz, 1H), 4.16-4.07 (m, 6H), 3.79-3.76 (m, 1H), 3.74 (t, J=6.5, Hz, 1H), 3.53-3.50 (m, 2H). ¹³C NMR (125 MHz, CDCl₃): δ 134.83, 134.52, 134.38, 117.80, 117.72, 117.38, 100.12, 76.57, 74.45, 74.33, 73.95, 72.56, 70.50, 70.40, 64.86. HRMS (DART-MS) m/z calcd. for C₁₅H₂₃O₅ [M+H]⁺ 283.15400. found: 283.15507.

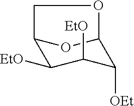 [SMILES: CCOC1C2COC(O2)C(OCC)C1OCC]

Prepared according to the general procedure using 1,6-anhydro-β-D-galactose (0.5 g, 3.1 mmol, 1.0 equiv), NaH (0.45 g, 10.8 mmol, 3.5 equiv), ethyl iodide (0.9 mL, 10.8 mmol, 3.5 equiv) in DMF (15 mL) at 25° C. The crude was purified by flash chromatography (hexane/ethyl acetate=6/1) to afford the desired product Et-D-Gal as a colorless liquid (0.64 g, 84% yield). ¹H NMR (500 MHZ, CDCl₃): δ 5.37 (s, 1H), 4.42-4.38 (m, 2H), 3.72-3.69 (m, 1H), 3.68-3.66 (m, 1H), 3.65-3.55 (m, 7H), 3.41-3.38 (m, 1H), 1.24-1.18 (m, 9H). ¹³C NMR (125 MHz, CDCl₃): δ 100.18, 77.80, 75.41, 73.50, 72.96, 66.97, 65.93, 64.56, 64.23, 15.50, 15.47, 15.45. HRMS (DART-MS) m/z calcd. for C₁₂H₂₃O₅ [M+H]⁺ 247.15400. found: 247.15430.

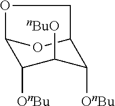

Prepared according to the general procedure using 1,6-anhydro-β-L-glucose⁴³ (1.0 g, 6.2 mmol, 1.0 equiv), NaH (1.1 g, 27.7 mmol, 4.5 equiv), 1-bromobutane (3.0 mL, 27.7 mmol, 4.5 equiv) in DMF (30 mL) at 50° C. The crude was purified by flash chromatography (hexane/ethyl acetate=12/1) to afford the product ⁿBu-L-Glc as a colorless liquid (1.2 g, 59% yield). ¹H NMR (500 MHZ, CDCl₃): δ 5.40 (s, 1H), 4.55 (dd, J=5.8, 1.4 Hz, 1H), 3.87 (dd, J=7.0, 1.2 Hz, 1H), 3.69 (dd, J=7.0, 5.8 Hz, 1H), 3.59-3.49 (m, 6H), 3.37-3.34 (m, 1H), 3.18-3.16 (m, 1H), 3.15-3.13 (m, 1H), 1.62-1.52 (m, 6H), 1.43-1.35 (m, 6H), 0.94-0.90 (m, 9H).

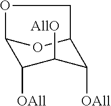

Prepared according to the general procedure using 1,6-anhydro-β-L-glucose⁴³ (0.82 g, 5.0 mmol, 1.0 equiv), NaH (0.7 g, 17.5 mmol, 3.5 equiv), allyl bromide (1.5 mL, 17.5 mmol, 3.5 equiv) in DMF (15 mL) at 25° C. The crude was purified by flash chromatography (hexane/ethyl acetate=6/1) to afford the product All-L-Glc as a colorless liquid (1.2 g, 85% yield). ¹H NMR (500 MHZ, CDCl₃): δ 5.98-5.86 (m, 3H), 5.43 (s, 1H), 5.33-5.27 (m, 3H), 5.22-5.19 (m, 3H), 4.58 (dd, J=5.8, 1.4 Hz, 1H), 4.14-4.09 (m, 6H), 3.92 (dd, J=7.2, 1.2 Hz, 1H), 3.70 (dd, J=7.2, 5.8 Hz, 1H), 3.52-3.50 (m, 1H), 3.32-3.30 (m, 1H), 3.28-3.26 (m, ¹H).

### Initiator Synthesis

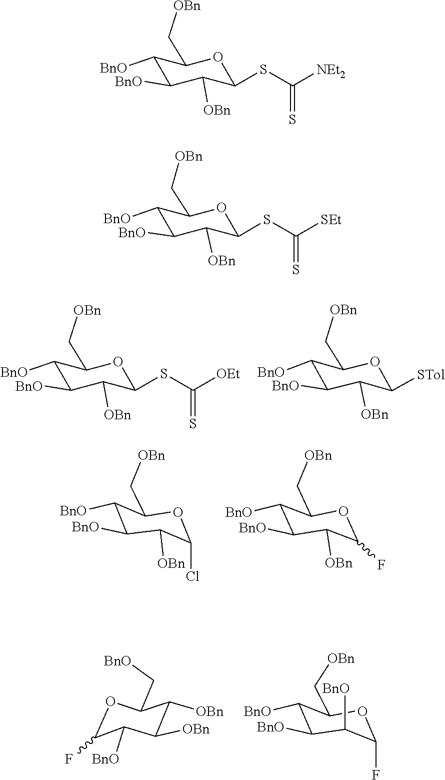

I-1, I-S2, I-S3, I-2 were prepared according to literature procedures⁴⁴⁻⁴⁷.

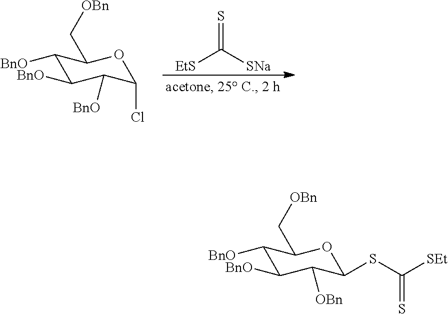

Literature procedures were modified for the preparation of I-S1⁴⁸. A solution of I-2 (450 mg, 0.8 mmol, 1.0 equiv) in acetone (3 mL) was added dropwise to the suspension of sodium ethyl trithiocarbonate (155 mg, 1.0 mmol, 1.2 equiv) in acetone (3 mL) over 10 mins at 25° C. The resulting mixture was stirred for 2 hrs, filtered through Celite, washed with ethyl acetate. The solvent was removed in vacuo and the crude was purified by flash chromatography (hexane/ethyl acetate=10/1) to afford the desired product I-S1 as a yellow syrup (400 mg, 76% yield). ¹H NMR (500 MHZ, CDCl₃) δ 7.38-7.25 (m, 18H), 7.19-7.15 (m, 2H), 5.70 (d, J=10.1 Hz, 1H), 4.90 (q, J=11.0 Hz, 2H), 4.82 (d, J=10.9 Hz, ¹H), 4.77 (s, 2H), 4.60 (dd, J=25.1, 11.4 Hz, 2H), 4.50 (d, J=12.1 Hz, 1H), 3.85-3.72 (m, 4H), 3.67 (dd, J=10.2, 8.4 Hz, 1H), 3.65-3.60 (m, 1H), 3.38 (q, J=7.4 Hz, 2H), 1.36 (t, J=7.5 Hz, 3H). ¹³C NMR (151 MHZ, CDCl₃): δ 220.70, 138.34, 138.05, 137.50, 128.43, 128.38, 128.31, 128.20, 127.93, 127.89, 127.80, 127.72, 127.69, 127.59, 86.92, 86.43, 79.77, 79.48, 77.58, 75.76, 75.54, 74.96, 73.43, 68.41, 31.39, 12.87. HRMS (DART-MS) m/z calcd. for C₃₇H₄₄NO₅S₃ [M+NH₄]⁺ 678.23761. found: 678.23755.

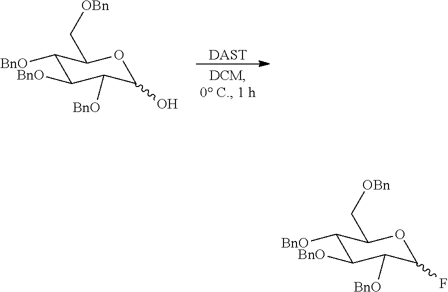 [SMILES: OC1OC(COCc2ccccc2)C(OCc2ccccc2)C(OCc2ccccc2)C1OCc1ccccc1 ; "DAST" ; FC1OC(COCc2ccccc2)C(OCc2ccccc2)C(OCc2ccccc2)C1OCc1ccccc1]

DAST (0.7 mL, 5.5 mmol, 1.1 equiv) was added dropwise over 10 min to a stirring solution of 2,3,4,6-tetra-O-benzyl-D-glucopyranose (2.7 g, 5.0 mmol, 1.0 equiv) in dry CH₂Cl₂ (25 mL) at 0° C., and the reaction was left stirring for 1 hr. Saturated aqueous NaHCO₃ (5 mL) was added, and the mixture was stirred vigorously for 15 mins. Then the organic phase was isolated and washed with brine, and the organic extract was dried over Na₂SO₄. After filtration, all the volatiles were removed under vacuum and purified by flash chromatography (hexane/ethyl acetate=10/1), yielding the desired product I-3 (2.5 g, 92% yield, α/β=1/4) as a colorless syrup. ¹H NMR for I-3 was essentially the same as reported previously⁴⁹.

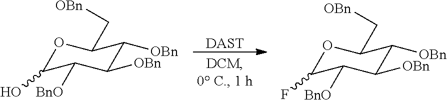

DAST (0.3 mL, 2.2 mmol, 1.1 equiv) was added dropwise over 10 min to a stirring solution of 2,3,4,6-tetra-O-benzyl-L-glucopyranose⁵⁰ (1.1 g, 2.0 mmol, 1.0 equiv) in dry CH₂Cl₂ (10 mL) at 0° C., and the reaction was left stirring for 1 hr. Saturated aqueous NaHCO₃ (5 mL) was added, and the mixture was stirred vigorously for 15 mins. Then the organic phase was isolated and washed with brine, and the organic extract was dried over Na₂SO₄. After filtration, all the volatiles were removed under vacuum and purified by flash chromatography (hexane/ethyl acetate=10/1), yielding the desired product ent-I-3 (1.0 g, 90% yield, α/β=1/2.2) as a colorless syrup. ¹H NMR for ent-I-3 was essentially the same as reported previously⁴⁹.

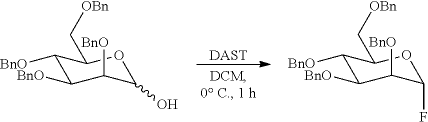 [SMILES: OC1OC(COCc2ccccc2)C(OCc2ccccc2)C(OCc2ccccc2)C1OCc1ccccc1 ; "DAST" ; FC1OC(COCc2ccccc2)C(OCc2ccccc2)C(OCc2ccccc2)C1OCc1ccccc1]

DAST (0.5 mL, 3.4 mmol, 1.1 equiv) was added dropwise over 10 min to a stirring solution of 2,3,4,6-tetra-O-benzyl-D-mannopyranose⁵⁰ (1.7 g, 3.1 mmol, 1.0 equiv) in dry CH₂Cl₂ (20 mL) at 0° C., and the reaction was left stirring for 1 hr. Saturated aqueous NaHCO₃ (5 mL) was added, and the mixture was stirred vigorously for 15 mins. Then the organic phase was isolated and washed with brine, and the organic extract was dried over Na₂SO₄. After filtration, all the volatiles were removed under vacuum and purified by flash chromatography (hexane/ethyl acetate=12/1), yielding the desired product I-4 (1.5 g, 88% yield) as a colorless syrup. ¹H NMR spectra is consistent with literature data⁵¹.

### Crosslinker Synthesis

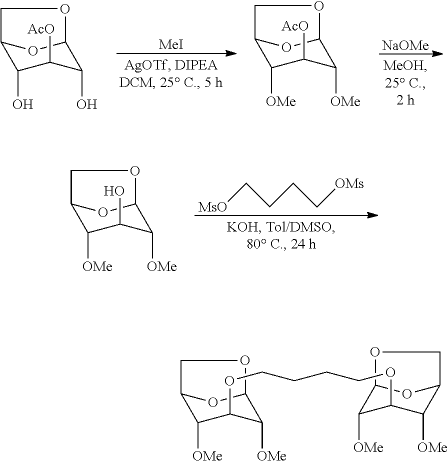

To a solution of CL-Me-S1⁵² (2.0 g, 9.8 mmol, 1.0 equiv) and silver triflate (10.1 g, 39.2 mmol, 4.0 equiv) in CH₂Cl₂ (70 mL) under a nitrogen atmosphere at 0° C., was added diisopropylethylamine (5.1 mL, 29.4 mmol, 3.0 equiv) slowly followed by methyl iodide (2.0 mL, 31.4 mmol, 3.2 equiv) dropwise. The reaction was stirred at 25° C. for 5 hrs, the brown reaction mixture was then filtered through a pad of silica gel, washing with ethyl acetate. The solvent was removed in vacuo and the crude was purified by flash chromatography (hexane/ethyl acetate=1/1) to afford the desired product CL-Me-S2 as a white solid (2.0 g, 88% yield). ¹H NMR spectra is consistent with literature data⁵³. ¹H NMR (500 MHZ, CDCl₃): δ 5.48 (s, 1H), 5.00-4.97 (m, 1H), 4.65 (dd, J=6.0, 1.4 Hz, 1H), 3.97 (dd, J=7.4, 1.2 Hz, 1H), 3.72 (dd, J=7.4, 6.0 Hz, 1H), 3.51 (s, 3H), 3.50 (s, 3H), 3.11-3.09 (m, 1H), 3.06-3.04 (m, 1H), 2.11 (s, 3H).

To a solution of CL-Me-S2 (2.0 g, 8.6 mmol, 1.0 equiv) in MeOH (20 mL) at 25° C., was added MeONa (46.5 mg, 0.86 mmol, 0.1 equiv). The reaction was stirred at 25° C. for 2 hrs and neutralized by Amberlite resin IR-120 (H⁺). After filtration, the solvent was removed in vacuo to furnish the desired product CL-Me-S3 as a colorless syrup, the syrup was used for next step without further purification.

Prepared according to a modified literature procedure⁵⁴. To a solution of 1,4-butanediol dimethanesulfonate (740 mg, 3.0 mmol, 1.0 equiv) and CL-Me-S3 (860 mg, 4.5 mmol, 1.5 equiv) in toluene (2.5 mL) and dimethyl sulfoxide (10 mL) was added KOH (675 mg, 12.0 mmol, 4.0 equiv). The reaction was stirred at 80° C. for 12 hrs, after cooled down to room temperature, the mixture was diluted with ethyl acetate. The organic layer was washed with brine and dried over Na₂SO₄. After filtration, the solvent was removed in vacuo and the crude was purified by flash chromatography (hexane/ethyl acetate=1/2) to afford the desired product CL-Me as a colorless syrup (800 mg, 81% yield). ¹H NMR (500 MHZ, CDCl₃): δ 5.46 (s, 2H), 4.62 (dd, J=6.0, 1.6 Hz, 2H), 3.93 (dd, J=7.2, 1.2 Hz, 2H), 3.71 (dd, J=7.2, 6.0 Hz, 2H), 3.61-3.51 (m, 4H), 3.46 (s, 6H), 3.45 (s, 6H), 3.41-3.39 (m, 2H), 3.13-3.11 (m, 2H), 3.09-3.07 (m, 2H), 1.67-1.63 (m, 4H). ¹³C NMR (125 MHz, CDCl₃): δ 99.87, 79.52, 78.61, 76.74, 73.10, 69.99, 65.14, 57.80, 57.12, 26.71. HRMS (DART-MS) m/z calcd. for C₂₀H₃₅O₁₀ [M+H]⁺ 435.22247. found: 435.22181.

### Polymer Synthesis

General procedure: In a glovebox under a nitrogen atmosphere, an oven-dried vial was charged with [M], [I] and DCM (or CDCl₃), BF₃·Et₂O was added. After 6 hrs, the reaction vial was removed from the glovebox and quenched with two drops of methanol. The polymer solution was precipitated into cold hexane or MeOH/H₂O (v/v, 1/1), centrifuged, discarded the solvent, and redissolve in CHCl₃. This procedure was repeated three times to ensure any catalyst residue or unreacted monomer was removed. The polymer was dried under high vacuum overnight to a constant weight.

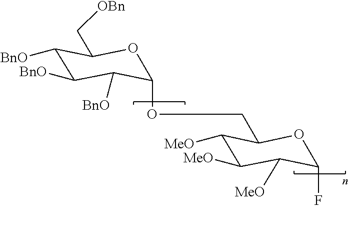

Prepared according to the general procedure using Me-D-Glc (510.6 mg, 2.5 mmol), I-3 (0.2 M, 250 μL, 50 μmol), CDCl₃ (488 μL) and BF₃·Et₂O (0.2 M, 12.5 μL, 2.5 μmol), the polymer solution was precipitated into cold hexane to give a fine white powder P(Me-D-Glc) (510.7 mg, 95% yield). ¹H NMR (600 MHZ, CDCl₃): δ 4.94 (d, J=3.8 Hz, ¹H), 3.83-3.76 (m, 1H), 3.70-3.62 (m, 1H), 3.58 (s, 3H), 3.51 (s, 3H), 3.48-3.45 (m, 1H), 3.43 (s, 3H), 3.20 (t, J=9.4 Hz, 1H), 3.11 (dd, J=9.6, 3.6 Hz, ¹H). ¹³C NMR (125 MHZ, CDCl₃): δ 96.34, 83.26, 81.72, 79.44, 70.18, 65.91, 60.62, 60.41, 58.17. ¹⁹F NMR (470 MHz, CDCl₃): δ −149.14 (dd, J=53.1, 25.7 Hz). SEC (CHCl₃): Mₙ=5.8 kDa, Ð=1.23. [α]_{D}²⁰=201.3 (c 1.08, CHCl₃).

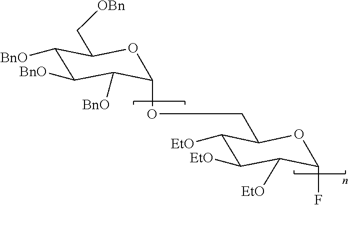

Prepared according to the general procedure using Et-D-Glc (49.3 mg, 48 μL, 0.2 mmol), I-3 (0.4 M, 5 μL, 2 μmol), CDCl₃ (70 μL) and BF₃·Et₂O (0.02 M, 10 μL, 0.2 μmol), the polymer solution was precipitated into cold hexane to give a fine white powder P(Et-D-Glc) (35.6 mg, 70% yield). ¹H NMR (600 MHZ, CDCl₃): δ 4.93 (d, J=3.6 Hz, 1H), 3.87-3.80 (m, 2H), 3.75-3.62 (m, 6H), 3.56-3.50 (m, 2H), 3.34 (t, J=9.4 Hz, 1H), 3.14 (dd, J=9.6, 3.4 Hz, 1H), 1.20-1.13 (m, 9H). ¹³C NMR (125 MHZ, CDCl₃): δ 96.99, 81.27, 80.33, 77.52, 70.73, 68.40, 68.13, 65.76, 65.43, 15.83, 15.81, 15.65. ¹⁹F NMR (470 MHZ, CDCl₃): δ −148.86 (dd, J=53.4, 25.7 Hz). SEC (CHCl₃): Mₙ=12.0 kDa, Ð=1.23. [α]_{D}²⁰=171.7 (c 1.21, CHCl₃).

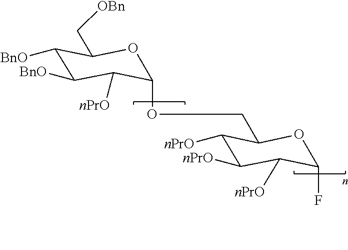

Prepared according to the general procedure using ⁿPr-D-Glc (86.5 mg, 84 μL, 0.3 mmol), I-3 (0.2 M, 15 μL, 3 μmol), DCM (42 μL) and BF₃·Et₂O (0.01 M, 9 μL, 0.09 μmol), the polymer solution was precipitated into MeOH/H₂O (v/v, 1/1) to give a fine white powder P(ⁿPr-D-Glc) (44.7 mg, 52% yield). ¹H NMR (600 MHZ, CDCl₃): δ 4.92 (d, J=3.4 Hz, 1H), 3.80-3.72 (m, 3H), 3.66-3.50 (m, 6H), 3.42-3.35 (m, 2H), 3.14 (dd, J=9.6, 3.4 Hz, 1H), 1.61-1.51 (m, 6H), 0.94-0.86 (m, 9H). ¹³C NMR (125 MHZ, CDCl₃): δ 96.99, 81.39, 80.71, 77.40, 74.88, 74.49, 72.08, 70.80, 65.44, 23.64, 23.50, 23.31, 10.62, 10.58, 10.55. ¹⁹F NMR (470 MHZ, CDCl₃): δ −151.44 (dd, J=53.5, 25.3 Hz). SEC (CHCl₃): Mₙ=16.7 kDa, Ð=1.38. [α]_{D}²⁰=146.1 (c 0.62, CHCl₃).

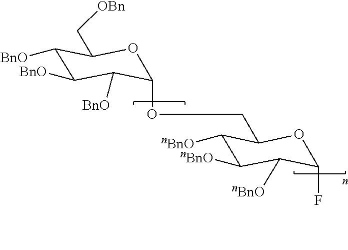

Prepared according to the general procedure using ⁿBu-D-Glc (99.1 mg, 96 μL, 0.3 mmol), I-3 (0.2 M, 15 μL, 3 μmol), tetrabutylammonium difluorotriphenylsilicate (0.001 M, 18 μL, 0.018 μmol), DCM (3 μL) and BF₃·Et₂O (0.01 M, 18 μL, 0.18 μmol), the polymer solution was precipitated into MeOH/H₂O (v/v, 1/1) to give a colorless syrup P(ⁿBu-D-Glc) (58.6 mg, 59% yield). ¹H NMR (600 MHZ, CDCl₃): δ 4.91 (d, J=3.4 Hz, 1H), 3.82-3.72 (m, 3H), 3.66-3.50 (m, 6H), 3.45-3.35 (m, 2H), 3.13 (dd, J=9.6, 3.4 Hz, 1H), 1.61-1.51 (m, 6H), 1.41-1.31 (m, 6H), 0.94-0.86 (m, 9H). ¹³C NMR (125 MHZ, CDCl₃): δ 97.14, 81.46, 80.79, 77.25, 73.03, 72.62, 70.86, 70.22, 65.47, 32.71, 32.53, 32.27, 19.37, 19.29, 19.28, 14.01, 13.98, 13.93. ¹⁹F NMR (470 MHZ, CDCl₃): δ −148.88 (dd, J=53.3, 26.0 Hz). SEC (CHCl₃): Mₙ=18.1 kDa, Ð=1.13. [α]_{D}²⁰=132.8 (c 0.95, CHCl₃).

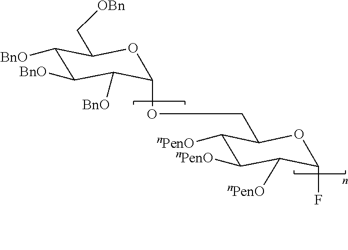

Prepared according to the general procedure using ⁿPen-D-Glc (111.8 mg, 108 μL, 0.3 mmol), I-3 (0.2 M, 15 μL, 3 μmol), tetrabutylammonium difluorotriphenylsilicate (0.001 M, 18 μL, 0.018 μmol) and BF₃·Et₂O (0.01 M, 18 μL, 0.18 μmol), the polymer solution was precipitated into MeOH/H₂O (v/v, 1/1) to give a colorless syrup P(ⁿPen-D-Glc) (69.4 mg, 62% yield). ¹H NMR (600 MHZ, CDCl₃): δ 4.91 (d, J=3.4 Hz, 1H), 3.82-3.72 (m, 3H), 3.66-3.50 (m, 6H), 3.45-3.35 (m, 2H), 3.13 (dd, J=9.6, 3.4 Hz, 1H), 1.63-1.45 (m, 6H), 1.37-1.25 (m, 12H), 0.92-0.86 (m, 9H). ¹³C NMR (125 MHZ, CDCl₃): δ 97.25, 81.43, 80.79, 77.12, 73.36, 72.87, 70.92, 70.48, 65.43, 30.28, 30.12, 29.86, 28.41, 28.29, 22.65, 22.58, 22.55, 14.04, 14.01. ¹⁹F NMR (470 MHZ, CDCl₃): δ −148.81 (dd, J=53.5, 25.9 Hz). SEC (CHCl₃): Mₙ=18.6 kDa, Ð=1.16. [α]_{D}²⁰=118.4 (c 1.13, CHCl₃).

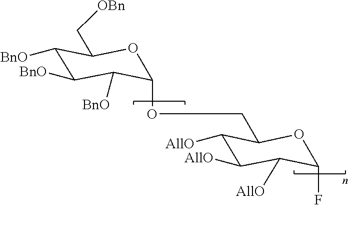

Prepared according to the general procedure using All-D-Glc (141.2 mg, 140 μL, 0.5 mmol), I-3 (0.4 M, 125 μL, 50 μmol) and BF₃·Et₂O (0.2 M, 4 μL, 0.8 μmol), the polymer solution was precipitated into MeOH/H₂O (v/v, 1/1) to give a colorless syrup P(All-D-Glc) (126.0 mg, 75% yield). ¹H NMR (600 MHZ, CDCl₃): δ 6.00-5.80 (m, 3H), 5.31-5.21 (m, 3H), 5.18-5.06 (m, 3H), 4.98-4.90 (m, 1H), 4.36-4.32 (m, 2H), 4.26-4.20 (m, 1H), 4.18-4.08 (m, 3H), 3.85-3.76 (m, 1H), 3.73-3.62 (m, 3H), 3.55-3.45 (m, 1H), 3.35-3.25 (m, ¹H). ¹³C NMR (125 MHZ, CDCl₃): δ 135.53, 135.15, 116.39, 116.08, 97.15, 81.13, 79.50, 77.13, 73.99, 73.68, 71.23, 70.70, 65.50. ¹⁹F NMR (470 MHZ, CDCl₃): δ −139.33 (dd, J=53.2, 12.9 Hz), −149.06 (dd, J=53.2, 25.5 Hz). SEC (CHCl₃): Mₙ=2.6 kDa, Ð=1.18. [α]_{D}²⁰=121.0 (c 1.67, CHCl₃).

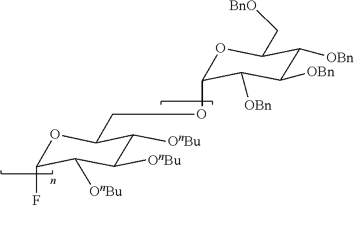

Prepared according to the general procedure using ⁿBu-L-Glc (66.1 mg, 68 μL, 0.2 mmol), ent-I-3 (0.2 M, 10 μL, 2 μmol), DCM (10 μL) and BF₃·Et₂O (0.02 M, 10 μL, 0.2 μmol), the polymer solution was precipitated into MeOH/H₂O (v/v, 1/1) to give a colorless syrup P(ⁿBu-L-Glc) (46.3 mg, 70% yield). ¹H NMR (600 MHZ, CDCl₃): δ 4.91 (d, J=3.4 Hz, 1H), 3.82-3.72 (m, 3H), 3.66-3.48 (m, 6H), 3.45-3.35 (m, 2H), 3.13 (dd, J=9.6, 3.4 Hz, 1H), 1.61-1.49 (m, 6H), 1.42-1.30 (m, 6H), 0.94-0.86 (m, 9H). ¹³C NMR (125 MHZ, CDCl₃): δ 97.13, 81.45, 80.78, 77.25, 73.03, 72.61, 70.85, 70.23, 65.46, 32.70, 32.53, 32.26, 19.36, 19.29, 19.27, 14.01, 13.98, 13.92. ¹⁹F NMR (470 MHZ, CDCl₃): δ −151.42 (dd, J=53.2, 25.8 Hz). SEC (CHCl₃): Mₙ=18.5 kDa, Ð=1.21. [α]_{D}²⁰=−133.9 (c 0.67, CHCl₃).

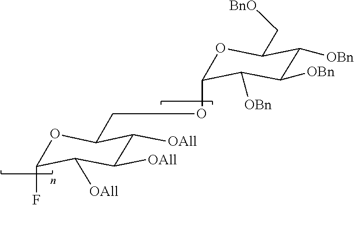

Prepared according to the general procedure using All-L-Glc (141.2 mg, 140 μL, 0.5 mmol), ent-I-3 (0.4 M, 125 μL, 50 μmol) and BF₃·Et₂O (0.2 M, 5 μL, 1.0 μmol), the polymer solution was precipitated into MeOH/H₂O (v/v, 1/1) to give a colorless syrup P(All-L-Glc) (131.1 mg, 78% yield). ¹H NMR (600 MHZ, CDCl₃): δ 6.00-5.80 (m, 3H), 5.31-5.21 (m, 3H), 5.18-5.06 (m, 3H), 4.98-4.90 (m, 1H), 4.36-4.32 (m, 2H), 4.26-4.21 (m, ¹H), 4.18-4.06 (m, 3H), 3.85-3.75 (m, 1H), 3.73-3.62 (m, 3H), 3.55-3.45 (m, 1H), 3.35-3.25 (m, ¹H). ¹³C NMR (125 MHZ, CDCl₃): δ 135.51, 135.13, 116.40, 116.09, 97.14, 81.11, 79.48, 77.10, 73.99, 73.67, 71.23, 70.68, 65.47. ¹⁹F NMR (470 MHZ, CDCl₃): δ −139.32 (dd, J=53.2, 12.9 Hz), −149.05 (dd, J=53.2, 25.5 Hz). SEC (CHCl₃): Mₙ=2.6 kDa, Ð=1.17. [α]_{D}²⁰=−123.4 (c 1.50, CHCl₃).

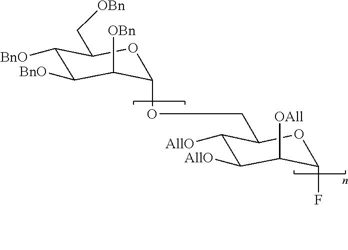

Prepared according to the general procedure using All-D-Man (56.5 mg, 56 μL, 0.2 mmol), 1-4 (0.4 M, 33.5 μL, 13.4 μmol) and BF₃·Et₂O (0.2 M, 2.5 μL, 0.5 μmol), the polymer solution was precipitated into MeOH/H₂O (v/v, 1/1) to give a colorless syrup P(All-D-Man) (51.9 mg, 81% yield). ¹H NMR (600 MHZ, CDCl₃): δ 5.98-5.79 (m, 3H), 5.35-5.07 (m, 6H), 5.00-4.87 (m, 1H), 4.41-4.34 (m, 1H), 4.20-4.00 (m, 6H), 3.90-3.86 (m, ¹H), 3.82-3.78 (m, 1H), 3.75-3.70 (m, 2H), 3.61-3.59 (m, ¹H). ¹³C NMR (125 MHz, CDCl₃): δ 135.25, 134.94, 134.52, 116.78, 116.63, 115.92, 98.62, 79.41, 75.23, 74.08, 73.72, 71.90, 71.61, 70.87, 66.12. ¹⁹F NMR (470 MHZ, CDCl₃): δ −138.04 (d, J=50.7 Hz). SEC (CHCl₃): Mₙ=3.3 kDa, Ð=1.24. [α]_{D}²⁰=131.1 (c 2.33, CHCl₃).

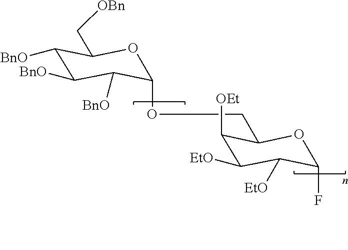

Prepared according to the general procedure using Et-D-Gal (24.6 mg, 24 μL, 0.1 mmol), I-3 (0.2 M, 10 μL, 2 μmol), TBAT (0.001 M, 6 μL, 0.006 μmol), DCM (7 μL) and BF₃·Et₂O (0.01 M, 4 μL, 0.04 μmol), the polymer solution was precipitated into cold hexane to give a fine white powder P(Et-D-Gal) (16.7 mg, 68% yield). ¹H NMR (600 MHZ, CDCl₃): δ 4.87-4.75 (m, 1H), 4.03-3.92 (m, 2H), 3.90-3.83 (m, 1H), 3.78-3.50 (m, 9H), 1.20-1.13 (m, 9H). ¹³C NMR (125 MHZ, CDCl₃): δ 98.53, 79.83, 75.53, 74.07, 71.76, 68.10, 66.23, 65.97, 65.42, 15.80, 15.62, 15.49. ¹⁹F NMR (470 MHz, CDCl₃): δ −150.17 (dd, J=53.9, 25.5 Hz). SEC (CHCl₃): Mₙ=6.4 kDa, Ð=1.14. [α]_{D}²⁰=143.2 (c 0.73, CHCl₃).

### Glycan Synthesis

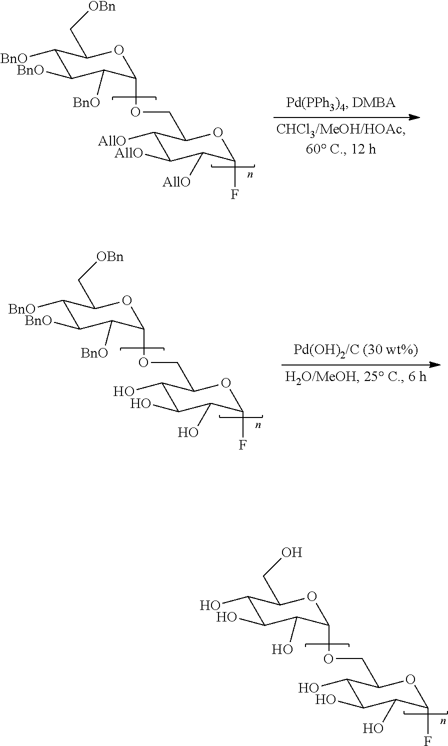

Prepared according to a modified literature procedure⁵⁵. To an oven-dried 10 mL Schlenk flask equipped with a stir bar was added P(All-D-Glc) (100 mg), 1,3-dimethylbarbituric acid (DMBA, 300 mg, 6 equiv), Pd(PPh₃)₄ (74 mg, 0.2 equiv), HOAc (120 μL), CHCl₃ (1.2 mL) and MeOH (2.4 mL). Then the reaction mixture was stirred vigorously for 12 hrs at 60° C. After cooled down to room temperature, the solvent was removed, H₂O (0.4 mL) was added. The mixture was precipitated into acetone, centrifuged, discarded the solvent, and redissolve in H₂O. This procedure was repeated three times, after that, the mixture was further purified by reverse phase column (20-80% MeOH/H₂O) to yield a white foam P(OH^{Bn}-D-Glc) after lyophilization. ¹H NMR (600 MHZ, D₂O): δ 4.99 (s, 1H), 4.05-3.96 (m, 1H), 3.95-3.89 (m, 1H), 3.80-3.70 (m, 2H), 3.63-3.57 (m, 1H), 3.55-3.50 (m, ¹H). ¹³C NMR (151 MHZ, D₂O): δ 97.62, 73.32, 71.32, 70.10, 69.45, 65.46. ¹⁹F NMR (470 MHZ, D₂O): δ −150.4-−151.2 (br). SEC (DMF): Mₙ=1.7 kDa, Ð=1.20. [α]_{D}²⁰=137.6 (c 0.093, H₂O).

The P(OH^{Bn}-D-Glc) (20 mg) was dissolved in a mixture of H₂O/MeOH (1 mL/1 mL) containing 20% Pd(OH)₂/C (6 mg, 30 wt %). The resulting mixture was stirred under H₂ atmosphere (1 atm) for 12 hrs at 25° C., and was then filtrated through a pad of Celite, washed with H₂O/MeOH (v/v, 1/1) three times. The filtrates were concentrated, and the crude was purified by reverse phase column (20-50% MeOH/H₂O) to yield a white foam P(OH-D-Glc) after lyophilization. ¹H NMR (600 MHZ, D₂O): δ 5.00-4.98 (m, 1H), 4.05-3.98 (m, 1H), 3.95-3.89 (m, 1H), 3.80-3.70 (m, 2H), 3.65-3.57 (m, 1H), 3.55-3.49 (m, ¹H). ¹³C NMR (125 MHZ, D₂O): δ 97.67, 73.36, 71.36, 70.15, 69.50, 65.52. ¹⁹F NMR (470 MHz, D₂O): δ −150.82 (dd, J=53.4, 26.5 Hz). [α]_{D}²⁰=150.8 (c 0.122, H₂O).

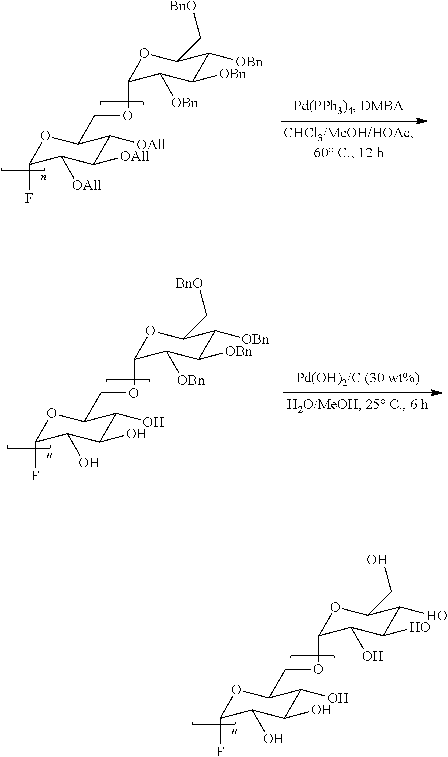

To an oven-dried 10 mL Schlenk flask equipped with a stir bar was added P(All-L-Glc) (100 mg), 1,3-dimethylbarbituric acid (DMBA, 300 mg, 6 equiv), Pd(PPh₃)₄ (74 mg, 0.2 equiv), HOAc (120 μL), CHCl₃ (1.2 mL) and MeOH (2.4 mL). Then the reaction mixture was stirred vigorously for 12 hrs at 60° C. After cooled down to room temperature, the solvent was removed, H₂O (0.4 mL) was added. The mixture was precipitated into acetone, centrifuged, discarded the solvent, and redissolve in H₂O. This procedure was repeated three times, after that, the mixture was further purified by reverse phase column (20-80% MeOH/H₂O) to yield a white foam P(OH^{Bn}-L-Glc) after lyophilization. ¹H NMR (600 MHZ, D₂O): δ 5.00 (s, 1H), 4.05-3.96 (m, 1H), 3.95-3.88 (m, 1H), 3.81-3.71 (m, 2H), 3.63-3.57 (m, 1H), 3.56-3.50 (m, ¹H). ¹³C NMR (151 MHz, D₂O): δ 97.66, 73.35, 71.35, 70.13, 69.49, 65.50. ¹⁹F NMR (470 MHZ, D₂O): δ −150.5-−151.0 (br). SEC (DMF): Mₙ=1.7 kDa, Ð=1.22. [α]_{D}²⁰=−137.6 (c 0.170, H₂O).

The P(OH^{Bn}-L-Glc) (20 mg) was dissolved in a mixture of H₂O/MeOH (1 mL/1 mL) containing 20% Pd(OH)₂/C (6 mg, 30 wt %). The resulting mixture was stirred under H₂ atmosphere (1 atm) for 12 hrs at 25° C., and was then filtrated through a pad of Celite, washed with H₂O/MeOH (v/v, 1/1) three times. The filtrates were concentrated, and the crude was purified by reverse phase column (20-50% MeOH/H₂O) to yield a white foam P(OH-L-Glc) after lyophilization. ¹H NMR (600 MHZ, D₂O): δ 5.01-4.96 (m, 1H), 4.04-3.97 (m, 1H), 3.96-3.88 (m, 1H), 3.80-3.71 (m, 2H), 3.62-3.57 (m, 1H), 3.56-3.51 (m, ¹H). ¹³C NMR (125 MHZ, D₂O): δ 97.67, 73.36, 71.37, 70.15, 69.50, 65.51. ¹⁹F NMR (470 MHz, D₂O): 0-150.83 (dd, J=53.2, 26.4 Hz). [α]_{D}²⁰=−149.4 (c 0.087, H₂O).

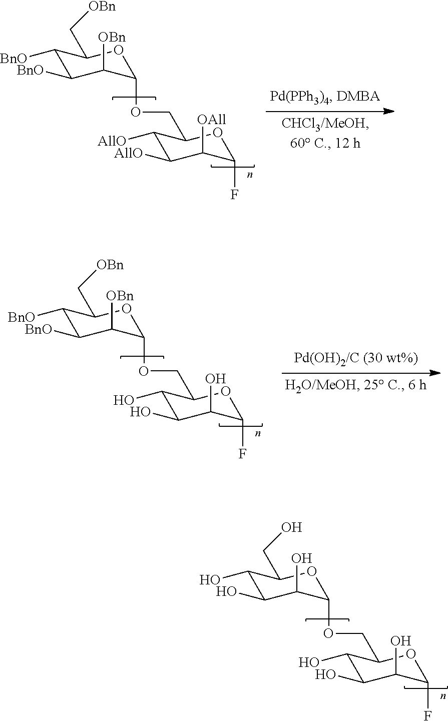

To an oven-dried 10 mL Schlenk flask equipped with a stir bar was added P(All-D-Man) (100 mg), 1,3-dimethylbarbituric acid (DMBA, 300 mg, 6 equiv), Pd(PPh₃)₄ (74 mg, 0.2 equiv), CHCl₃ (1.2 mL) and MeOH (2.4 mL). Then the reaction mixture was stirred vigorously for 12 hrs at 60° C. After cooled down to room temperature, the solvent was removed, H₂O (0.4 mL) was added. The mixture was precipitated into acetone, centrifuged, discarded the solvent, and redissolve in H₂O. This procedure was repeated three times, after that, the mixture was further purified by reverse phase column (20-80% MeOH/H₂O) to yield a white foam P(OH^{Bn}-D-Man) after lyophilization. ¹H NMR (600 MHZ, D₂O): δ 4.92 (s, 1H), 4.05-3.70 (m, 6H). ¹³C NMR (125 MHZ, D₂O): δ 99.31, 70.80, 70.66, 69.95, 66.58, 65.49. ¹⁹F NMR (470 MHZ, D₂O): δ −150.5-−151.0 (br). SEC (DMF): Mₙ=2.0 kDa, Ð=1.26. [α]_{D}²⁰=45.3 (c 0.053, H₂O).

The P(OH^{Bn}-D-Man) (20 mg) was dissolved in a mixture of H₂O/MeOH (1 mL/1 mL) containing 20% Pd(OH)₂/C (6 mg, 30 wt %). The resulting mixture was stirred under H₂ atmosphere (1 atm) for 12 hrs at 25° C., and was then filtrated through a pad of Celite, washed with H₂O/MeOH (v/v, 1/1) three times. The filtrates were concentrated, and the crude was purified by reverse phase column (20-50% MeOH/H₂O) to yield a white foam P(OH-D-Man) after lyophilization. ¹H NMR (600 MHZ, D₂O): δ 4.94-4.88 (m, 1H), 4.01 (s, ¹H), 3.98-3.91 (m, 1H), 3.90-3.83 (m, 2H), 3.82-3.78 (m, 1H), 3.76-3.70 (m, ¹H). ¹³C NMR (125 MHZ, D₂O): δ 99.32, 70.81, 70.67, 69.96, 66.58, 65.49. ¹⁹F NMR (470 MHZ, D₂O): δ −139.00 (d, J=47.3 Hz). [α]_{D}²⁰=60.0 (c 0.080, H₂O).

### Chain Extensions

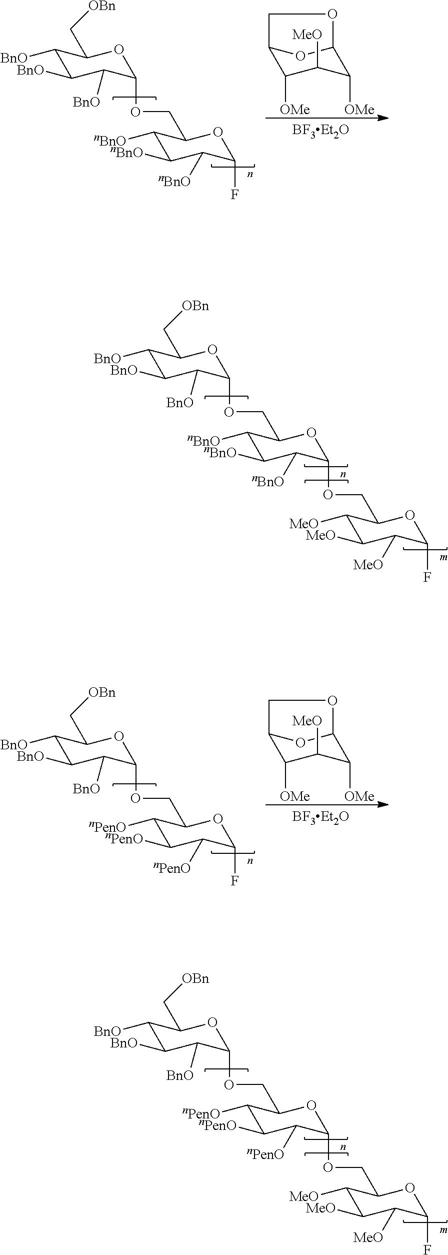

P(ⁿBu-D-Glc) was prepared according to the general procedure with [M]₀:[I]₀=33. The macroinitiator P(ⁿBu-D-Glc) (7 mg) and Me-D-Glc (10.2 mg) was dissolve in CDCl₃ (40 μL), BF₃·Et₂O (0.2 M, 0.6 μL, 0.12 μmol) was added. The polymer solution was precipitated into MeOH/H₂O (v/v, 1/1) to give a diblock copolysaccharide P(ⁿBu-D-Glc)-b-P(Me-D-Glc) as a white powder. P(ⁿPen-D-Glc)-b-P(Me-D-Glc) was prepared through a similar procedure, except P(ⁿPen-D-Glc) was used as a macroinitiator. The successful chain extension was confirmed by a complete shift to higher molecular weight, a monomodal distribution with low D and little to no tailing in SEC traces, as well as a single peak in the diffusion-ordered spectroscopy (DOSY) NMR analysis.

### Chain End Modifications

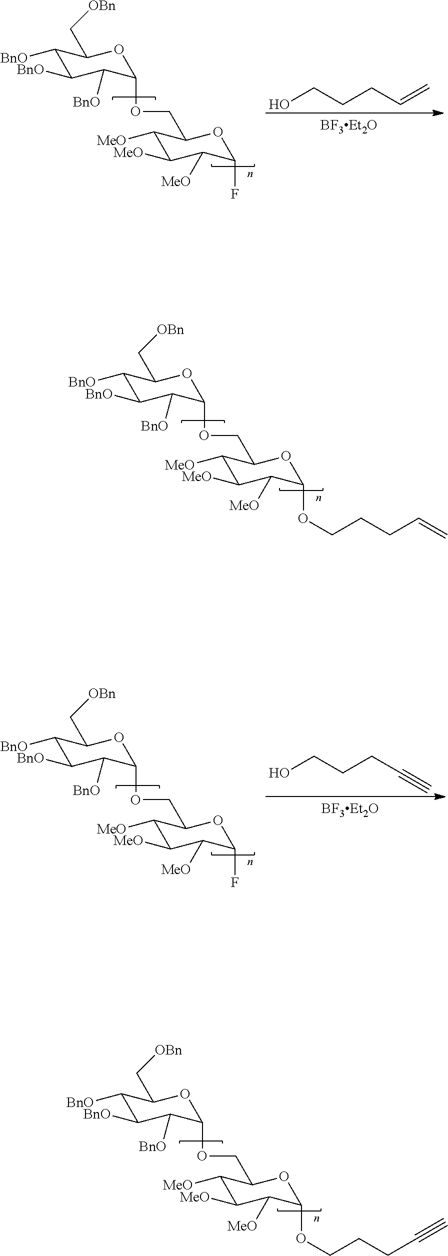

P(Me-D-Glc) was prepared according to the general procedure with [M]₀:[I]₀=15. P(Me-D-Glc) (34 mg) was dissolved in CDCl₃ (0.2 mL), alkenyl alcohol (0.5 M, 27 μL, 13.5 μmol) and BF₃·Et₂O (0.2 M, 25 μL, 5 μmol) was sequentially added. After 1 hr, the reaction was quenched with two drops of methanol. The polymer solution was precipitated into cold hexane to give P(Me^{O1}-D-Glc) as a white powder. P(Me^{O2}-D-Glc) was prepared through a similar procedure, except alkynyl was used as a nucleophile. The ¹H-NMR and MALDI-TOF MS analyses indicated that the transformations of ω-F chain end were quantitative.

### Enzymatic Degradation Studies

Adapted from a literature report⁵⁶: P(OH^{Bn}-D-Glc) or P(OH^{Bn}-L-Glc) solutions (1.0 mg, 10 mg/mL) in ammonium acetate (100 μL, 5 mM, pH 5.5) were degraded by dextranase (from Penicillium funiculosum, 0.5 U/mL) at 37° C. for 72 hrs. The mixtures were lyophilized to provide white foams. The molecular weights were determined by SEC using DMF (0.01 M LiBr) as the eluent. SEC analyses revealed complete degradation of D-glucan, while L-glucan remained intact.

### Chemical Resistance Studies

P(Me-D-Glc) (50 mg, Mₙ=7.8 kDa, Ð=1.23) was dissolved in CHCl₃ (2.0 mL), additive (5 equiv relative to repeating units) was added. After 24 hrs, the polymer solution was precipitated into cold methanol to remove additive. SEC analysis show little to no change on Mₙ and dispersity for the polymer treated by various additives.

### Thermodynamic Studies

In a glovebox under a nitrogen atmosphere, an oven-dried J. Young NMR tube was charged with Et-D-Glc (200 μL, 0.8 mmol), I-3 (0.2 M, 40 μL, 8 μmol) and 282 μL of CDCl₃. BF₃·Et₂O (0.2 M, 8 μL, 1.6 μmol) was added ([Et-D-Glc]₀=1.5 M), the tube was sealed and inverted three times to ensure adequate mixing of the reagents. The NMR tube was then taken out of the glovebox and brought into a 600 MHZ NMR probe preheated to the desired polymerization temperature (25, 33, 40, 50 and 60° C., respectively). The polymerization was monitored by ¹H NMR (no. scans=8, delay=5 s) at different time intervals until the conversion remained constant at each temperature. The equilibrium monomer concentration, [Et-D-Glc]_{eq}, was calculated to be 0.090, 0.120, 0.180, 0.225 and 0.330 M for 25, 33, 40, 50 and 60° C., respectively (Table 5), based on the equation [Et-D-Glc]_{eq}=[Et-D-Glc]₀×(1−conv.). The thermodynamic parameters were calculated to be ΔH=−26.8 kJ·mol⁻¹ and ΔS=−69.7 J·mol⁻¹·K⁻¹ by the linear fitting of the plot of Rln[Et-D-Glc]_{eq} vs. 1/T according to Van't Hoff equation: Rln[Et-D-Glc]_{eq}=ΔH/T−ΔS. Therefore, the ceiling temperature was calculated to be T_{c}=93° C. for [Et-D-Glc]₀=1.0 M, based on the equation T_{c}^{o}=ΔH^{o}/ΔS^{o}=ΔH/(ΔS−Rln[Et-D-Glc]₀), assuming ΔH=ΔH^{o}

### Depolymerization Studies

### Depolymerzation of P(Me-D-Glc) Under Thermolysis Condition

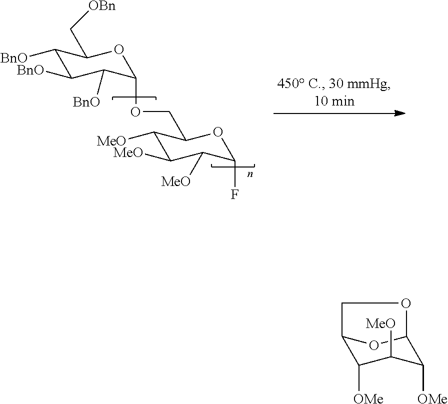

To an oven-dried 5 mL Schlenk flask equipped with a condenser tube and a receiving flask was added P(Me-D-Glc) (93.0 mg), the system was then connected to house vacuum (30 mmHg). The reaction was heated for 10 mins at 450° C. After cooled down to room temperature, the liquid in the condenser tube and receiving flask was collected, rinsed with CHCl₃. All the volatiles were removed under vacuum and the crude was purified by flash chromatography to afford a light-yellow liquid (31.6 mg, 34% yield), ¹H NMR analysis showed that monomer Me-D-Glc was recovered in a pure state.

### Depolymerization of P(Et-D-Glc) Under Chemolysis Condition

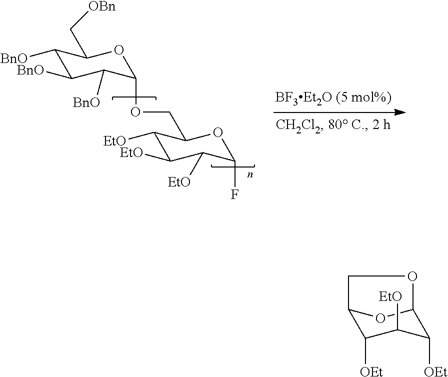

An oven-dried 50 mL Schlenk flask was charged with P(Et-D-Glc) (400 mg), CH₂Cl₂ (15 mL) and BF₃·Et₂O (7.5 μL, 60 μmol, 5 mol %) was added. The reaction mixture was heated for 2 hrs at 80° C. After cooled down to room temperature, the resulting mixture was then filtered through a pad of silica gel, washing with ethyl acetate. The solvent was removed in vacuo and the crude was purified by flash chromatography (hexane/ethyl acetate=2/1) to afford the desired product Et-D-Glc as a light-yellow liquid (346 mg, 86% yield). ¹H NMR analysis showed that monomer Et-D-Glc was recovered in a pure state.

### Depolymerization of Bulk Materials (PN1, PN2) Under Chemolysis Condition

An oven-dried 250 mL Schlenk flask was charged with bulk materials PN1 and PN2 (6.0 g), CH₂Cl₂ (120 mL) and BF₃·Et₂O (150 μL, 1.2 mmol, 5 mol %) was added. The reaction mixture was heated for 2 hrs at 80° C. After cooled down to room temperature, the resulting mixture was then filtered through a pad of silica gel, washing with ethyl acetate. The solvent was removed in vacuo and the crude was purified by flash chromatography (hexane/ethyl acetate=10/1 to 2/1 to 1/2) to afford the desired product Me-D-Glc (1.76 g), ⁿPen-D-Glc (2.71 g) and CL-Me (0.60 g). ¹H NMR analysis showed that both monomers and crosslinker was recovered in a pure state, and the recycle yield was estimated to be 85%. In addition, the recovered anhydrosugars could be repolymerized to give PN1′/PN2′ with identical tensile properties to pristine material.

### Polymer Film Preparation

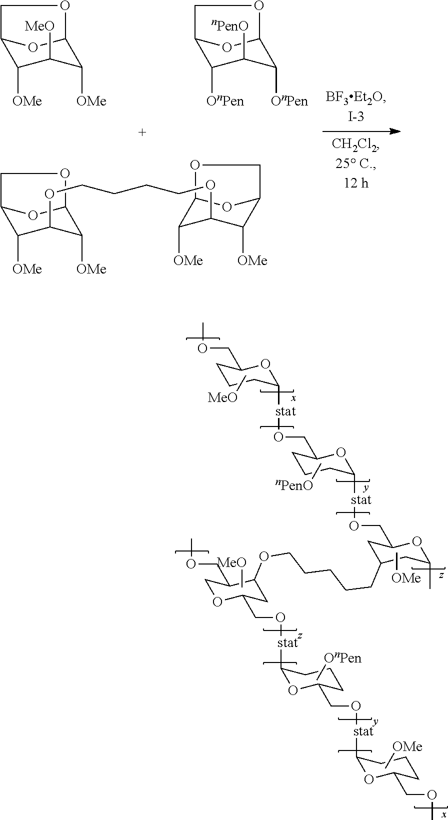

General procedure: In a glovebox under a nitrogen atmosphere, an oven-dried vial was charged with Me-D-Glc, ⁿPen-D-Glc, CL-Me and I-3. To this solution was added BF₃·Et₂O, and the mixture was transferred into a dog-bone mold (dimensions: 63.5×9.5×3.2 mm³). The sample was allowed to cure at room temperature for 12 hrs. The specimen was carefully removed from the mold, the residual monomer and catalyst were removed by Soxhlet extraction with MeOH. The sample was dried under high vacuum overnight to a constant weight.

PN1/PN1′ was prepared according to the general procedure using Me-D-Glc (114.4 mg, 560 μmol), ⁿPen-D-Glc (357.6 mg, 384 μL, 960 μmol), CL-Me (1.0 M, 80 μL, 80 μmol), I-3 (0.2 M, 80 μL, 16 μmol) and BF₃·Et₂O (0.2 M, 70 μL, 14 μmol).

PN2/PN2′ was prepared according to the general procedure using Me-D-Glc (196.1 mg, 960 μmol), ⁿPen-D-Glc (208.6 mg, 224 μL, 560 μmol), CL-Me (1.0 M, 80 μL, 80 μmol), I-3 (0.2 M, 80 μL, 16 μmol), DCM (110 μL) and BF₃·Et₂O (0.2 M, 50 μL, 10 μmol).

### Conditions Optimization

General procedure: In a glovebox under a nitrogen atmosphere, an oven-dried vial was charged with Me-D-Glc (20.5 mg, 0.1 mmol), I-3 (0.2 M, 10 μL, 2 μmol), and solution of Lewis acid in DCM or CH₃CN was added to give a final reaction concentration of 2.0 M. After 3 hrs, the reaction vial was removed from the glovebox and quenched with two drops of methanol. The polymer solution was precipitated into cold hexane, centrifuged, discarded the solvent, and redissolve in CHCl₃. This procedure was repeated three times to ensure any catalyst residue or unreacted monomer was removed. The polymer was dried under high vacuum overnight to a constant weight. The experimental results were summarized in Table 1 below.

### Example 2

### Preparation and Characterization of Precision Polysaccharides

From the outset, O-methylated 1,6-anhydro-b-D-glucopyranose Me-D-Glc was utilized as a model monomer, which can be readily obtained after a one-step methylation of the biomass-derived and commercially available 1,6-anhydro-b-D-glucopyranose ($1.3/g) in high yield and decagram scale (94%, 11.8 g). Previous works by Kamigaito²¹ and Fors²² have shown that thiocarbamate chain transfer agents could regulate the propagation of oxocarbenium ions, which are structurally like the proposed propagating species in the polymerization of 1,6-anhydrosugars (FIG. 1c). Separately, glycosyl thiocarbamate also served as a good glycosyl donor in catalytic glycosylation reactions²³. Based on these seminal reports, a series of glycosyl donors were evaluated, including glycosyl thiocarbamate I-1, glycosyl trithiocarbonate I-S1, and glycosyl xanthate I-S2, for their ability to initiate the CROP of 1,6-anhydrosugars by mild Lewis acid catalysts (Table 1).

Table 1. Summary of conditions optimization results for Me-D-Glc. Monomer conversion was determined by ¹H NMR spectroscopy; M_{n,GPC} and Ð were determined by SEC analysis calibrated to polystyrene standard; w-chain end fidelity was determined by NMR analysis using w-fidelity=I^{5.55-5.66 ppm}/(I^{7.10-7.12 ppm}/2), where I^{5.55-5.66 ppm} and I^{7.10-7.12 ppm} correspond to integrals of signal at 5.55-5.66 ppm and 7.10-7.12 ppm attributed to hydrogen of the w-chain end and two hydrogens of the a-chain end, respectively.

TABLE 1

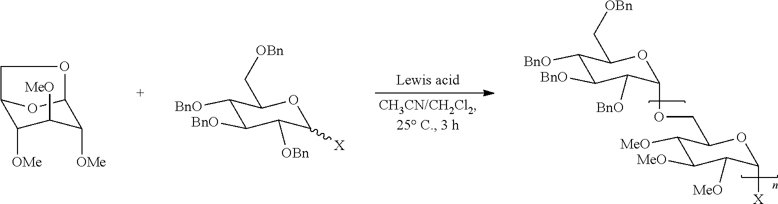

| Entry | Initiator | Lewis acid | [M]/[I]/[LA] | Conversion (%) | M_{n,GPC}(kDa) | Ð | ω-fidelity (%) |
|---|---|---|---|---|---|---|---|
| 1 | I-1 | SnCl₄ | 50/1/0.1 | 0 | — | — | — |
| 2 | I-1 | SbCl₅ | 50/1/0.1 | 0 | — | — | — |
| 3 | I-1 | BF₃•Et₂O | 50/1/0.1 | 0 | — | — | — |
| 4 | I-1 | DMTSF | 50/1/0.1 | 0 | — | — | — |
| 5 | I-1 | SbF₅ | 50/1/0.1 | 15 | — | — | 0 |
| 6 | I-1 | AgOTf | 50/1/0.5 | 30 | — | — | 0 |
| 7 | I-1 | Cu(OTf)₂ | 50/1/0.5 | 88 | 6.9 | 1.54 | 3 |
| 8 | I-S1 | Cu(OTf)₂ | 50/1/0.5 | 95 | 5.7 | 1.73 | 0 |
| 9 | I-S2 | Cu(OTf)₂ | 50/1/0.5 | 96 | 5.2 | 1.74 | 0 |
| 10 | I-S3 | Cu(OTf)₂ | 50/1/0.5 | 39 | 5.5 | 1.56 | 0 |
| 11 | I-2 | Cu(OTf)₂ | 50/1/0.5 | 95 | — | — | 60 |
| 12 | I-2 | Al(OTf)₃ | 50/1/0.5 | 30 | — | — | 0 |
| 13 | I-3 | Cu(OTf)₂ | 50/1/0.5 | 50 | — | — | 0 |
| 14 | I-3 | AgOTf | 50/1/0.5 | 0 | — | — | — |
| 15 | I-3 | ZnCl₂ | 50/1/0.5 | 0 | — | — | — |
| 16 | I-3 | BF₃•Et₂O | 50/1/0.05 | 80 | 5.8 | 1.23 | 90 |

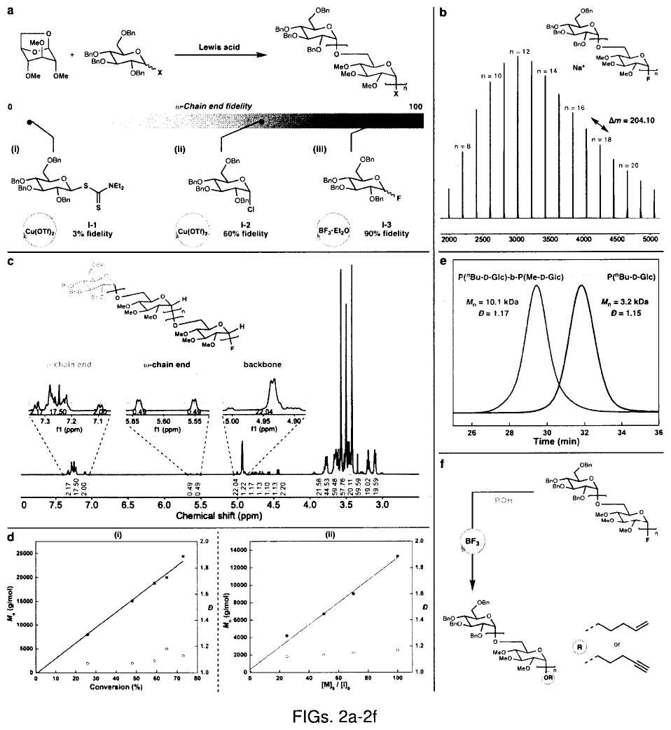

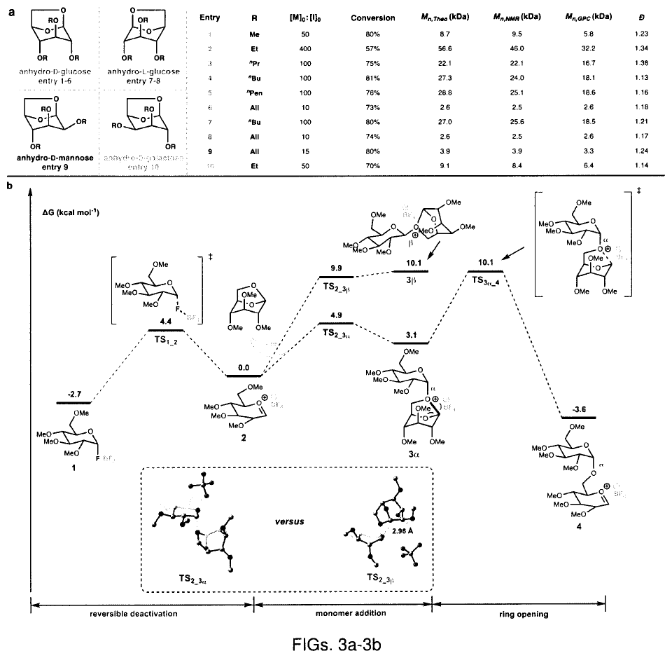

While I-1 in conjunction with Cu(OTf)₂ produced a polysaccharide with a number-average molecular weight (Mₙ) of 6.9 kg/mol, the high dispersity (Ð=1.54) and the low fidelity of the w-chain end suggested poor control over the polymerization (FIG. 2a (i)). The low level of control may be attributed to the lability of C—S bond in these glycosyl donors under acidic condition and that more stable glycosyl donors could lead to improved control. Indeed, glycosyl chloride I-2 was found to afford a polysaccharide with a 60% fidelity of the w-chain end (FIG. 2a (ii)). Encouragingly, a more stable glycosyl fluoride 1-3 provided an excellent w-chain end fidelity (90%) when boron trifluoride etherate (BF₃·Et₂O) was used as the catalyst (FIG. 2a (iii)). Size-exclusion chromatography (SEC) analysis of the sample, P(Me-D-Glc), revealed a monomodal molar mass distribution with a dispersity (D) of 1.23. The number-average molecular weight (Mₙ) calculated based on the ¹H NMR analysis (9.5 kg/mol) agreed well with the theoretical value (8.7 kg/mol) (FIG. 3a, entry 1). In addition, the chain-end groups were confirmed by matrix assisted laser desorption/ionization-time-of-flight (MALDI-TOF) mass spectroscopy (FIG. 2b). The well-defined structure of P(Me-D-Glc), including the a-, w-chain ends and a-1,6-glucan backbone, was further demonstrated by ¹H NMR (FIG. 2c) and ¹⁹F NMR analyses. Notably, the single anomeric carbon signal at 96.34 ppm in ¹³C NMR and the high positive specific rotation of [α]_{D}²⁰=+201.3° unambiguously supported that P(Me-D-Glc) was highly stereoregular²⁴, i.e., a-glycosidic linkages were exclusively generated. The polymerization also displayed characteristics consistent with living polymerization, including first-order reaction kinetics, linear growth of the molecular weights over conversion, controlled molecular weights proportional to [M]₀/[I]₀ ratio, and low dispersity (FIG. 2d).

### Example 2

### Monomer Scope

With the initiator/catalyst pair identified, the monomer scope of this polymerization was explored (FIG. 3a). O-alkylated 1,6-anhydrosugars with various alkyl side chains, including ethyl, n-propyl, n-butyl, n-pentyl, and allyl all exhibited good reactivities and excellent control over the polymerization (FIG. 3a, entry 2-6). Notably, ¹H, ¹³C NMR, and high positive specific rotation values consistently supported exclusive a-1,6-D-glycosidic linkages in these polysaccharides. Remarkably, a polysaccharide with a degree of polymerization (DP) of 185 was obtained with relatively low dispersity (Ð=1.38) when O-ethyl monomer Et-D-Glc was polymerized (FIG. 3a, entry 2). For the 1,6-anhydrosugars with long O-alkyl side chains (e.g., n-butyl and n-pentyl), the addition of external fluoride ions further improved control over the polymerization (FIG. 3a, entry 4-5). Furthermore, this methodology was not limited to 1,6-anhydroglucose. O-allyl 1,6-anhydromannose All-D-Man and O-ethyl 1,6-anhydrogalactose Et-D-Gal could also be polymerized with high efficiency and excellent control (FIG. 3a, entry 9-10), yielding well-defined a-1,6-polymannose and a-1,6-polygalactose, respectively.

Table 2. Summary of polymerization results for various monomers. The polymerizations were performed at 25° C. in dichloromethane for 6 hrs; monomer conversion was determined by ¹H NMR spectroscopy; M_{n,Theo} was calculated using the following equation: M_{n,Theo}=[M]₀/[I]₀×MW^{M}×conv.+MW^{I}, where MW^{M} and MW^{I} correspond to molar mass of monomer and initiator, respectively; M_{n,NMR} was calculated using the following equation: M_{n,NMR}=DP×MW^{M}+MW^{I}, and DP was determined by NMR analysis using the following equation: DP=I^{4.90-5.00 ppm}/(I^{7.10-7.12 ppm}/2), where I^{4.90-5.00 ppm} and I^{7.10-7.12 ppm} correspond to integrals of signal at 4.90-5.00 ppm and 7.10-7.12 ppm attributed to anomeric hydrogen of the polymer backbone and two hydrogens of the a-chain end, respectively; M_{n,GPC} and Ð were determined by SEC analysis calibrated to polystyrene standard.

TABLE 2

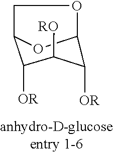

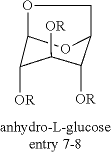

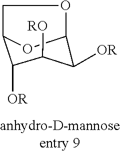

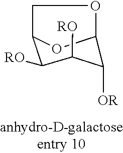

| Entry | Monomers | R | [M]₀: [I]₀ | Conversion | Mₙ,_{Theo}(kDa) | M_{n,NMR}(kDa) | M_{n,GPC}(kDa) | Ð |
|---|---|---|---|---|---|---|---|---|
| 1 | | Me | 50 | 80% | 8.7 | 9.5 | 5.8 | 1.23 |
| 2 | | Et | 400 | 57% | 56.6 | 46.0 | 32.2 | 1.34 |
| 3 | from D-glucose | ⁿPr | 100 | 75% | 22.1 | 22.1 | 16.7 | 1.38 |
| 4 | | ⁿBu | 100 | 81% | 27.3 | 24.0 | 18.1 | 1.13 |
| 5 | | ⁿPen | 100 | 76% | 28.8 | 25.1 | 18.6 | 1.16 |
| 6 | | All | 10 | 73% | 2.6 | 2.5 | 2.6 | 1.18 |
| 7 | from L-glucose | ⁿBu | 100 | 80% | 27.0 | 25.6 | 18.5 | 1.21 |
| 8 | | All | 10 | 74% | 2.6 | 2.5 | 2.6 | 1.17 |
| 9 | from D-mannose | All | 15 | 80% | 3.9 | 3.9 | 3.3 | 1.24 |
| 10 | from D-galactose | Et | 50 | 70% | 9.1 | 8.4 | 6.4 | 1.14 |

### Example 3

### Computational Investigations

Density functional theory (DFT) calculations were performed to gain more mechanistic insights into the reversible deactivation process in the living CROP and the origin of the excellent a-selectivity during the propagation step (FIG. 3b). The energy surface of activation of the dormant glycosyl fluoride 1 was first calculated, showing that the reaction readily gave an active oxocarbenium species 2 with an energy barrier of 7.1 kcal mol⁻¹. The facile activation of glycosyl fluoride chain end was attributed to the hardness of the fluorine atom, making it more readily to be abstracted by hard Lewis acid boron trifluoride. On the other hand, the reverse reaction, namely the deactivation of oxocarbenium species 2 by BF₄_{^{−}} counterion, occurred with a low energy barrier of 4.4 kcal mol⁻¹, and the fast deactivation process might arise from the strong anomeric effect of the glycosyl fluoride. In short, the synergy of glycosyl fluoride and boron trifluoride results in an equilibrium between the active and dormant species, with the ratio of propagating oxocarbenium:dormant species being 0.01. The low concentration of the propagating oxocarbenium results in livingness in the polymerization.

As for the propagation process, two sequential steps were then considered. A monomer first attacked the oxocarbenium to form an oxonium species 3, and then a subsequent ring opening reaction occurred to regenerate the propagating oxocarbenium. According to the DFT calculations, the energy barrier of the a-addition was lower than that of the b-addition (TS_{2_3a}=4.9 kcal mol⁻¹ versus TS_{2_3b}=9.9 kcal mol⁻¹). The difference in transition state energies is primarily attributed to the steric repulsion between the incoming anhydrosugar monomer and oxocarbenium species. This a-selectivity is consistent with the experimental results, where the single anomeric carbon signal was observed in ¹³C NMR spectra of the precision polysaccharides. A ring opening process was identified after the formation of oxonium intermediate 3a, which is considerably exergonic by 6.7 kcal mol⁻¹, indicative of the facile regeneration of the oxocarbenium. It is noteworthy that the direct addition to oxonium 3a by the anhydrosguar monomer proposed by Schuerch¹⁷ yielded no computationally viable transition states despite extensive searches. This result is consistent with a recent work by Reineke¹⁹.

### Example 4

### Chain Extension and Chain End Modifications

Synthesizing advanced polymeric architectures, such as block copolymers, represents one of the greatest advantages of living polymerization. Building upon this method, block copolysaccharides that are otherwise laborious to synthesize were accessed. First, polymerization of ⁿBu-D-Glc was carried out in the presence of I-3 to give a macroinitiator P(ⁿBu-D-Glc) (Mₙ=3.2 kg/mol, Ð=1.15) with an excellent fidelity of the w-chain end. Next, chain extension of P(ⁿBu-D-Glc) by Me-D-Glc yielded a diblock copolysaccharide P(ⁿBu-D-Glc)-b-P(Me-D-Glc). A shift to higher molecular weight, monomodal distribution with low Ð (1.17) and little to no tailing in SEC traces (FIG. 2e), as well as a single peak in the diffusion-ordered spectroscopy (DOSY) NMR analysis firmly supported the successful chain-extension.

Furthermore, as glycosyl fluoride has been widely used as a glycosyl donor in catalytic glycosylation reactions, the w-F chain end presents a versatile handle for further functionalization. As a proof of this principle, glycosylation of alkenyl and alkynyl alcohol by the w-F chain end of P(Me-D-Glc) was examined using BF₃·Et₂O as an activator (FIG. 2f). The ¹H NMR and MALDI-TOF MS analyses indicated that the transformations of w-F chain end were quantitative, creating an opportunity for the post-polymerization functionalization of precision polysaccharides via thiol-ene and Cu(I)-catalyzed azide-alkyne cycloaddition (CuAAC) click chemistries.

### Example 5

### Synthesis of Native Polysaccharides

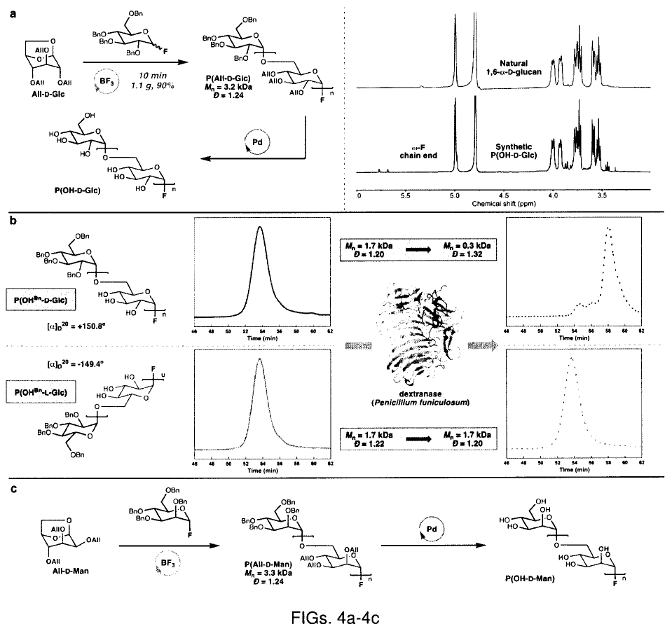

Oligomeric a-1,6-glycans play significant roles in many biological processes²⁵. Therefore, the current polymerization method was applied to access these biologically important glycans. Because of the facile removal of allyl groups, O-allyl 1,6-D-anhydroglucose All-D-Glc was chosen as the monomer. Polymerization of All-D-Glc at [M]₀:[I]₀ ratio of 10:1 yielded a precision polysaccharide P(All-D-Glc) with excellent molecular weight control (FIG. 3a, entry 6). Notably, this synthetic procedure was readily scalable (FIG. 4a): 1.1 grams of precise polysaccharide P(All-D-Glc) was obtained without loss of control as indicated by a monomodal SEC peak and low Ð (1.24). Followed by Pd-catalyzed deprotection, a well-defined a-1,6-D-glucan P(OH-D-Glc) was prepared. The obtained a-1,6-D-glucan showed identical ¹H NMR and ¹³C NMR spectra to the natural a-1,6-D-glucan (FIG. 4a), which further verified the excellent regio- and stereoselectivity of this method. Importantly, the w-F chain end was still observed in synthetic glucan, which could potentially be used as a glycosyl donor in enzymatic glycosylation to access more complex glycans²⁶.

Biosynthetic pathways of polysaccharides predominantly produce D-enantiomers which are susceptible to enzymatic degradation²⁷. In contrast, a chemical polymerization process does not discriminate either D- or L-anhydrosugars, providing a good opportunity to generate physiologically stable all-L-polysaccharides. Indeed, in the presence of a L-glycosyl fluoride initiator ent-I-3, 1,6-anhydro-b-L-glucopyranose ⁿBu-L-Glc and All-L-Glc could be polymerized to give the corresponding all-L-polysaccharide without any erosion in efficiency and livingness (FIG. 3a, entry 7-8). Applying the same deprotection protocol to P(All-L-Glc), a rare a-1,6-L-glucan was obtained. Equal but opposite-in-sign specific rotation values were observed for D- and L-glucans (FIG. 4b), confirming the stereoregularity and enantiopurity of both polysaccharides. To the best of our knowledge, the a-1,6-L-glucan prepared herein represents the first example of precision all-L-glycans. Both D- and L-glucans were treated with dextranase from Penicillium funiculosum. SEC analyses of the degradation reaction revealed complete degradation of D-glucan, while L-glucan remained intact (FIG. 4b). The remarkable resistance of L-glucans against glycosidase-mediated degradation suggested their potential physiological stability for future biomedical applications.

Furthermore, it was demonstrated the synthetic utility of the current living CROP strategy to synthesize a-1,6-D-mannan, a polysaccharide that exists in the cell wall of Mycobacterium tuberculosis and is implicated in the human immune response to pathogens²⁸. To produce precision a-1,6-D-mannan, All-D-Man was first polymerized into a well-defined polysaccharide P(All-D-Man) (Mₙ=3.3 kDa, Ð=1.24). After removing the allyl groups, a well-defined a-1,6-D-mannan was readily generated (FIG. 4c), which was determined to be composed of a-1,6-D-glycosidic linkages by ¹H and ¹³C NMR. Therefore, the current polymerization pathway becomes a promising alternative to the labor/equipment-demanding stepwise glycan assembly strategies for constructing biologically relevant polysaccharides²⁹.

### Example 6

### Depolymerization and Repolymerization

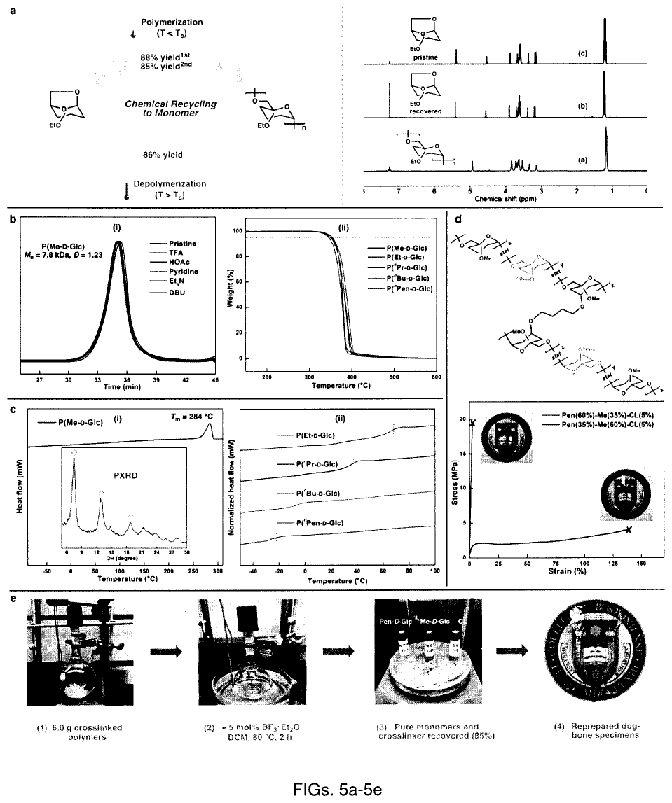

Beyond biologically active polysaccharides, renewable materials were also explored based on precision polysaccharides. One of the most desired properties of the next-generation sustainable polymers is their inherent chemical recyclability, which enables a closed-loop life cycle³⁰⁻³³. Given that 1,6-anhydroglucose was produced by the thermolysis of starch or cellulose, precision polysaccharide P(Me-D-Glc) was first subjected to thermolysis conditions (450° C., 30 mmHg). Indeed, monomer Me-D-Glc could be recovered in 34% yield. Despite the low yield, this result suggested the chemical recyclability of precision polysaccharides. Further thermodynamic analysis via variable-temperature ¹H NMR revealed the standard state thermodynamic parameters of the living CROP of Et-D-Glc: ΔH^{o}=−26.8 KJ/mol, ΔS^{o}=−73.1 J·mol⁻¹·K⁻¹. Correspondingly, the ceiling temperature (T_{c}) was calculated to be 366 K (93° C.) at [M]₀=1.0 M, suggesting that the precision polysaccharide could be catalytically depolymerized under a relatively mild condition close to the T_{c}. Consistent with this rationale, it was found that P(Et-D-Glc) could be quantitatively depolymerized to yield the monomer Et-D-Glc at 80° C. in the presence of a catalytic amount of BF₃·Et₂O (FIG. 5a). Additionally, the recovered Et-D-Glc was repolymerized to give P(Et-D-Glc) with an efficiency comparable to the pristine monomer. Therefore, a circular monomer-polymer-monomer life cycle of the precision polysaccharides was demonstrated (FIG. 5a).

Table 3. Mₙ vS [Et-D-Glc]₀:[I-3]₀ results. Experiments were performed according to general polymerization procedure, [Et-D-Glc]₀ was held constant and [I-3]₀ varied to achieve desired [Et-D-Glc]₀:[I-3]₀.

TABLE 3

| Entry | [M] | [M]₀:[I]₀ | Conversion | Mₙ_{,} _{Theo}(kDa) | Mₙ_{,} _{NMR}(kDa) | Mₙ_{,} _{GPC}(kDa) | Ð | [α]_{D}²⁰ |
|---|---|---|---|---|---|---|---|---|
| 1 | Et-D-Glc | 25 | 75% | 5.2 | 5.5 | 4.2 | 1.12 | +171.6 |
| 2 | Et-D-Glc | 50 | 79% | 10.2 | 10.4 | 6.7 | 1.13 | +171.1 |
| 3 | Et-D-Glc | 71 | 75% | 13.6 | 13.3 | 9.0 | 1.15 | +169.7 |
| 4 | Et-D-Glc | 100 | 74% | 18.0 | 18.7 | 13.3 | 1.17 | +172.4 |

Table 4. Kinetic studies for [Et-D-Glc] polymerization. Experiments were performed according to general polymerization procedure, [Et-D-Glc]₀=1.5 M, [Et-D-Glc]₀:[I-3]₀=200. Time-point aliquots were taken during polymerization for ¹H NMR and SEC analyses.

TABLE 4

| Entry | [M] | time | Conversion | Mₙ_{,} _{Theo}(kDa) | Mₙ_{,} _{NMR}(kDa) | Mₙ_{,} _{GPC}(kDa) | Ð |
|---|---|---|---|---|---|---|---|
| 1 | Et-D-Glc | 0.5 h | 27% | 13.8 | 13.3 | 8.0 | 1.07 |
| 2 | Et-D-Glc | 1.0 h | 48% | 24.2 | 22.7 | 15.1 | 1.07 |
| 3 | Et-D-Glc | 1.5 h | 59% | 29.6 | 25.4 | 18.8 | 1.09 |
| 4 | Et-D-Glc | 2.0 h | 65% | 32.5 | 31.3 | 20.0 | 1.18 |
| 5 | Et-D-Glc | 2.5 h | 74% | 37.0 | 35.0 | 24.4 | 1.13 |

TABLE 5
Summary of [Et-D-Glc]_{eq} obtained by VT-NMR.

| Entry | Temperature (° C.) | Conversion (%) | [Et-D-Glc]_{eq} (M) | Rln[Et-D-Glc]_{eq} (J · mol⁻¹ · K⁻¹) | 1/T (K⁻¹) |
|---|---|---|---|---|---|
| 1 | 25 | 94 | 0.090 | −20.0209 | 0.00335 |
| 2 | 33 | 92 | 0.120 | −17.6289 | 0.00327 |
| 3 | 40 | 88 | 0.180 | −14.2577 | 0.00314 |
| 4 | 50 | 85 | 0.225 | −12.4024 | 0.00305 |
| 5 | 60 | 78 | 0.330 | −9.2180 | 0.00296 |

### Example 7

### Material Properties

Labile main-chain groups (e.g., acetal, ester, enamine) are often incorporated into the existing chemically recyclable polymers to enable depolymerization^{20,34-38}, rendering these polymers unstable under harsh conditions (e.g., strong acid/base, high temperature, etc.). In contrast, the precision polysaccharides exhibited remarkable chemical and thermal stability. Little to no change in molecular weight and dispersity was found when exposing P(Me-D-Glc) to an excessive amount of Brønsted acid (e.g., acetic acid or trifluoroacetic acid) or base (e.g., pyridine, triethylamine, or 1,8-diazabicyclo[5.4.0]undec-7-ene) for 24 h (FIG. 5b (i)). Thermogravimetric analysis (TGA) revealed an onset decomposition temperature Ta (defined by 5% weight loss) greater than 345° C. (FIG. 5b (ii)). Impressively, the morphology and thermal performance of precision polysaccharides were readily tunable by side chain modifications. P(Me-D-Glc) turned out to be highly crystalline with well-defined diffraction patterns as revealed by powder X-ray diffraction (PXRD) spectrum, in which three major diffraction signals at 7.5°, 13.1°, and 18.9° were observed (FIG. 5c (i), inset). Additionally, a high melting temperature (Tₘ) of 284° C. was detected in differential scanning calorimetry (DSC) analysis (FIG. 5c (i)). In contrast, a Tₘ was not found in the polysaccharides carrying longer alkyl groups. At the same time, their glass-transition temperatures (T_{g}s) changed from 67° C. to −23° C. when varying the O-alkyl substitution from ethyl to n-pentyl (FIG. 5c (ii)). The precision polysaccharides represent a rare example that significant changes in the polymer morphology and thermal properties can be induced by simply altering the side chains ^{19,39}.

The chemical recyclability, together with good chemical and thermal stabilities make these polysaccharides promising candidates for sustainable materials (FIG. 5d). In particular, a copolysaccharide PN1 consisting of 65 mol % ⁿPen-D-Glc, 30 mol % Me-D-Glc, and 5 mol % crosslinker CL displayed mechanical properties typical for an elastomer: a Young's modulus of 106.63±16.17 MPa, a tensile strength of 4.06±0.09 MPa, and elongation at break reaching 138±15%. In contrast, a completely different material (PN2) was obtained when the copolysaccharide composition changed to 30 mol % ⁿPen-D-Glc, 65 mol % Me-D-Glc, and 5 mol % crosslinker CL. As a glassy material, PN2 showed higher Young's modulus (0.82±0.10 GPa) and ultimate tensile strength (19.17±1.92 MPa), with elongation at break of 2.6±0.3%. The drastic change in the mechanical properties was attributed to the different T_{g}s of PN1 and PN2 (22° C. vs. 55° C.)⁴⁰, which lead to elastic (PN1) or glassy (PN2) characteristics, respectively. Both copolysaccharide-based materials could be depolymerized completely (FIG. 5e), with the monomers and crosslinker recovered in excellent yield (85%). The recovered 1,6-anhydrosugars were repolymerized, and the resulting copolysaccharides PN1′ and PN2′ showed identical tensile properties to pristine PN1 and PN2, respectively.

Table 6. Stress, strain, and Young's modulus values of tensile tests at a strain rate of 5 mm/min for PN1 and PN1′ (sample 4).

TABLE 6

| Sample Number | Stress (MPa) | Strain (%) | E′ (MPa) |
|---|---|---|---|
| 1 | 4.09 | 148 | 96.48 |
| 2 | 4.10 | 132 | 107.86 |
| 3 | 4.00 | 133 | 115.55 |
| 4 | 4.17 | 167 | 86.03 |

Table 7. Stress, strain, and Young's modulus values of tensile tests at a strain rate of 5 mm/min for PN2 and PN2′ (sample 4).

TABLE 7

| Sample Number | Stress (MPa) | Strain (%) | E′ (GPa) |
|---|---|---|---|
| 1 | 19.36 | 2.5 | 0.81 |
| 2 | 17.95 | 2.4 | 0.77 |
| 3 | 20.21 | 2.8 | 0.89 |
| 4 | 17.13 | 1.8 | 1.01 |

### REFERENCE

1. Varki, A. et al. Essentials of Glycobiology (Cold Spring Harbor Laboratory Press, New York, ed. 2, 2009).
2. Ragauskas, A. J. et al. The path forward for biofuels and biomaterials. Science 311, 484-489 (2006).
3. Zhu, Y., Romain, C. & Williams, C. K. Sustainable polymers from renewable resources. Nature 540, 354-362 (2016).
4. Dumitriu, S. Polysaccharides: Structural Diversity and Functional Versatility (Marcel Dekker, New York, ed. 2, 2005).
5. Fittolani, G., Tyrikos-Ergas, T., Vargová, D., Chaube, M. A. & Delbianco, M. Progress and challenges in the synthesis of sequence controlled polysaccharides. Beilstein J. Org. Chem. 17, 1981-2025 (2021).
6. Kadokawa, J. Precision polysaccharide synthesis catalyzed by enzymes. Chem. Rev. 111, 4308-4345 (2011).
7. Zhu, Q. et al. Chemical synthesis of glycans up to a 128-mer relevant to the O-antigen of bacteroides vulgatus. Nat. Commun. 11, 4142-4148 (2020).
8. Guberman, M. & Seeberger, P. H. Automated glycan assembly: a perspective. J. Am. Chem. Soc. 141, 5581-5592 (2019).
9. Panza, M., Pistorio, S. G., Stine, K. J. & Demchenko, A. V. Automated chemical oligosaccharide synthesis: novel approach to traditional challenges. Chem. Rev. 118, 8105-8150 (2018).
10. Xiao, R. X. & Grinstaff, M. W. Chemical synthesis of polysaccharides and polysaccharide mimetics. Prog. Polym. Sci. 74, 78-116 (2017).
11. Saxon, D. J. et al. Architectural control of isosorbide-based polyethers via ring-opening polymerization. J. Am. Chem. Soc. 141, 5107-5111 (2019).
12. McGuire, T. M. et al. Control of crystallinity and stereocomplexation of synthetic carbohydrate polymers from D- and L-xylose. Angew. Chem., Int. Ed. 60, 4524-4528 (2021).
13. Stubbs, C. J. et al. Sugar-based polymers with stereochemistry-dependent degradability and mechanical properties. J. Am. Chem. Soc. 144, 1243-1250 (2022).
14. Debsharma, T., Yagci, Y. & Schlaad, H. Cellulose-derived functional polyacetal by cationic ring-opening polymerization of levoglucosenyl methyl ether. Angew. Chem., Int. Ed. 58, 18492-18495 (2019).
15. Mikami, K. et al. Polycarbonates derived from glucose via an organocatalytic approach. J. Am. Chem. Soc. 135, 6826-6829 (2013).
16. Dane, E. L. & Grinstaff, M. W. Poly-amido-saccharides: synthesis via anionic polymerization of a β-lactam sugar monomer. J. Am. Chem. Soc. 134, 16255-16264 (2012).
17. Ruckel, E. R. & Schuerch, C. Preparation of high polymers from 1,6-anhydro-2,3,4-tri-O-substituted b-D-glucopyranose. J. Org. Chem. 31, 2233-2239 (1966).
18. Aoshima, S. & Kanaoka, S. A renaissance in living cationic polymerization. Chem. Rev. 109, 5245-5287 (2009).
19. Porwal, M. K. et al. Stereoregular functionalized polysaccharides via cationic ring-opening polymerization of biomass derived levoglucosan. Chem. Sci. 13, 4512-4522 (2022).
20. Abel, B. A., Snyder, R. L. & Coates, G. W. Chemically recyclable thermoplastics from reversible-deactivation polymerization of cyclic acetals. Science 373, 783-789 (2021).
21. Uchiyama, M., Satoh, K. & Kamigaito, M. Cationic RAFT polymerization using ppm concentrations of organic acid. Angew. Chem., Int. Ed. 54, 1924-1928 (2015).
22. Kottisch, V., Michaudel, Q. & Fors, B. P. Cationic polymerization of vinyl ethers controlled by visible light. J. Am. Chem. Soc. 138, 15535-15538 (2016).
23. Nielsen, M. M. & Pedersen, C. M. Catalytic glycosylations in oligosaccharide synthesis. Chem. Rev. 118, 8285-8358 (2018).
24. Yoshida, D. & Yoshida, T. Elucidation of high ring-opening polymerizability of methylated 1,6-anhydro glucose. J. Polym. Sci. A. Polym. Chem. 47, 1013-1022 (2009).
25. Zhu, Q. Q. et al. Structural identification of (1→6)-a-D-glucan, a key responsible for the health benefits of longan, and evaluation of anticancer activity. Biomacromolecules 14, 1999-2003 (2013).
26. Williams, S. J. & Withers, S. G. Glycosyl fluorides in enzymatic reactions. Carbohydr. Res. 327, 27-46 (2000).
27. Stone, B. A., Svensson, B., Collins, M. E. & Rastall, R. A. Polysaccharide Degradation in Glycoscience (Springer, Heidelberg, ed. 2, 2008), pp. 2325-2375.
28. Apostolou, I. et al. Murine natural killer cells contribute to the granulomatous reaction caused by mycobacterial cell walls. Proc. Natl. Acad. Sci. U.S.A. 96, 5141-5146 (1999).
29. Calin, O., Eller, S. & Seeberger, P. H. Automated polysaccharide synthesis: assembly of a 30mer mannoside. Angew. Chem., Int. Ed. 52, 5862-5865 (2013).
30. Getzler, Y. D. Y. L. & Coates, G. W. Chemical recycling to monomer for an ideal, circular polymer economy. Nat. Rev. Mater. 5, 501-516 (2020).
31. Shi, C. et al. Design principles for intrinsically circular polymers with tunable properties. Chem 7, 2896-2912 (2021).
32. Mohadjer Beromi, M. et al. Iron-catalysed synthesis and chemical recycling of telechelic 1,3-enchained oligocyclobutanes. Nat. Chem. 13, 156-162 (2021).
33. Sathe, D. et al. Olefin metathesis-based chemically recyclable polymers enabled by fused-ring monomers. Nat. Chem. 13, 743-750 (2021).
34. Hong, M. & Chen, E. Y.-X. Completely recyclable biopolymers with linear and cyclic topologies via ring-opening polymerization of g-butyrolactone. Nat. Chem. 8, 42-49 (2016).
35. Zhu, J. B., Watson, E. M., Tang, J. & Chen, E. Y.-X. A synthetic polymer system with repeatable chemical recyclability. Science 360, 398-403 (2018).
36. Häußler, M., Eck, M., Rothauer, D. & Mecking, S. Closed-loop recycling of polyethylene-like materials. Nature 590, 423-427 (2021).
37. Yuan, J. S. et al. J. Am. Chem. Soc. 141, 4928-4935 (2019).
38. Christensen, P. R., Scheuermann, A. M., Loeffler, K. E. & Helms, B. A. Closed-loop recycling of plastics enabled by dynamic covalent diketoenamine bonds. Nat. Chem. 11, 442-448 (2019).
39. Song, Y. et al. Advancing the development of highly-functionalizable glucose-based polycarbonates by tuning of the glass transition temperature. J. Am. Chem. Soc. 140, 16053-16057 (2018).
40. Sangroniz, A. et al. Packaging materials with desired mechanical and barrier properties and full chemical recyclability. Nat. Commun. 10, 3559-3565 (2019).
41. Fu, L., Li, L. et al. Synthesis of clickable amphiphilic polysaccharides as nanoscopic assemblies. Chem. Commun. 50, 12742-12745 (2014).
42. Strašák, T. et al. Synthesis of substituted titanocene dichloride derivatives by hydrosilylation. J. Organomet. Chem. 768, 115-120 (2014).
43. Oikawa, M., Shintaku, T., Sekljic, H., Fukase, K. & Kusumoto, S. Synthesis of ¹³C-labeled biosynthetic precursor of lipid A and its analogue with shorter acyl chains. Bull. Chem. Soc. Jpn. 72, 1857-1867 (1999).
44. Padungros, P., Alberch, L. & Wei, A. Glycosyl dithiocarbamates: 3-selective couplings without auxiliary groups. J. Org. Chem. 79, 2611-2624 (2014).
45. Szeja, W. & Bogusiak, J. Synthesis of glycosyl xanthates from reducing sugar derivatives under phase-transfer conditions. Carbohydr. Res. 170, 235-239 (1987).
46. Veleti, S. K., Lindenberger, J. J., Thanna, S., Ronning, D. R. & Sucheck, S. J. Synthesis of a poly-hydroxypyrolidine-based inhibitor of Mycobacterium tuberculosis GlgE. J. Org. Chem. 79, 9444-9450 (2014).
47. Gómez, A. M., Pedregosa, A.; Casillas, M., Uriel, C. & López, J. C. Synthesis of C-1 alkyl and aryl glycals from pyranosyl or furanosyl chlorides by treatment with organolithium reagents. Eur. J. Org. Chem. 3579-3588 (2009).
48. Michaudel, Q. et al. Mechanistic insight into the photocontrolled cationic polymerization of vinyl ethers. J. Am. Chem. Soc. 139, 15530-15538 (2017).
49. L'Heureux, A. et al. Aminodifluorosulfinium salts: selective fluorination reagents with enhanced thermal stability and ease of handling. J. Org. Chem. 75, 3401-3411 (2010).
50. Chang, C.-W. et al. Establishment of guidelines for the control of glycosylation reactions and intermediates by quantitative assessment of reactivity. Angew. Chem., Int. Ed. 58, 16775-16779 (2019).
51. López, J. C., Bernal-Albert, P., Uriel, C., Valverde, S. & Gómez, A. M. IPy₂BF₄/HF-pyridine: a new combination of reagents for the transformation of partially unprotected thioglycosides and n-pentenyl glycosides to glycosyl fluorides. J. Org. Chem. 72, 10268-10271 (2007).
52. Su, X. B. et al. Synthesis of biaryl-containing medium-ring systems by organocuprate oxidation: applications in the total synthesis of ellagitannin natural products. Synthesis, 3880-3896 (2009).
53. Wollwage, P. C. & Seib, P. A. Preparation and proton magnetic resonance spectra of the methyl ethers of 1,6-anhydro-β-D-glucopyranose. J. Chem. Soc. C. 3143-3155 (1971).
54. Gouéth, P., Ramiz, A., Ronco, G., Mackenzie, G. & Villa, P. Synthesis of novel bis (glycosyl) ethers as bolaamphiphile surfactants. Carbohydr. Res. 266, 171-189 (1995).
55. Tsukamoto, H., Suzuki, T. & Kondo, Y. Remarkable solvent effect on Pd(0)-catalyzed deprotection of allyl ethers using barbituric acid derivatives: application to selective and successive removal of allyl, methallyl, and prenyl ethers. Synlett 3131-3136 (2007).
56. Franssen, O., van Ooijen, R. D., de Boer, D., Maes, R. A. A. & Hennink, W. E. Enzymatic degradation of cross-linked dextrans. Macromolecules 32, 2896-2902 (1999).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is:

1. A method of controlled synthesizing and chemical recycling of a stereoregular 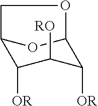
polysaccharide comprising reacting a biomass-derived anhydrosugar having the formula:
wherein R is a group selected from C1-C6 alkyl group,
with an initiator in the presence of a synergy of a catalyst and the initiator.

2. The method of claim 1, wherein the stereoregular polysaccharide is substantially converted to the anhydrosugar of Formula I by heating the stereoregular polysaccharide in the presence of the catalyst.

3. The method of claim 1, wherein the initiator is glucosyl halide having a structure represented by the formula: 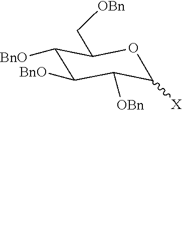
wherein the halide is a group selected from fluoride, chloride, bromide or iodide.

4. The method of claim 1, wherein the catalyst is a Lewis acid.

5. The method of claim 4, wherein the Lewis acid is Cu(OTf)₂, BF₃ or Boron trifluoride complex.

6. The method of claim 5, wherein the Boron trifluoride complex is boron trifluoride diethyl etherate.

7. The method of claim 1, wherein R is a group selected from C1-C7 alkyl group.

8. The method of claim 1, wherein R is a group selected from C1-C4 alkyl group.

9. The method of claim 1, wherein R is a group selected from C1-C3 alkyl group.

10. The method of claim 1, wherein R is a group selected from C1-C2 alkyl group.

11. The method of claim 1, wherein R is a methyl group.

12. The method of claim 1, wherein the initiator provides the polysaccharide with high chain end fidelity and an excellent molecular weight distribution in a controlled manner.

13. The method of claim 12, wherein the polysaccharide has 1-95% chain end fidelity.

14. The method of claim 12, wherein the polysaccharide has 50-95% chain end fidelity.

15. The method of claim 12, wherein the polysaccharide has about 90-95% chain end fidelity.

16. The method of claim 12, wherein a molecular weight of the polysaccharide ranges from 2.6 to 32.2 kDa.

17. A stereoregular polysaccharide prepared by the method of claim 1.
